(19) United States
(12) Patent Application Publication
(10) Pub. No.: US 2006/0276527 A1
(43) Pub. Date: Dec. 7, 2006

(54) Combination therapies for the treatment of cancer

(75) Inventor: George Tidmarsh, Portola Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Thresold Pharmaceuticals, Inc., Redwood City, CA (US)

(21) Appl. No.: 10/543,228

(22) PCT Filed: Jan. 16, 2004
(86) PCT No.: PCT/US04/01138

§ 371 Date: Jun. 22, 2006

Related U.S. Application Data
(60) Provisional application No. 60/441,110, filed on Jan. 17, 2003.
(60) Provisional application No. 60/442,344, filed on Jan. 23, 2003.
(60) Provisional application No. 60/458,663, filed on Mar. 28, 2003.

(51) Int. Cl. A61K 31/416

(52) U.S. Cl. 514/406

(57) ABSTRACT

Lonidamine or a lonidamine analog is administered with one or more additional anti-cancer agents or surgery or radiation to treat cancer or is administered alone or in combination to treat cancer, optionally in a sustained release formulation, and improve patient outcome.

### CROSS-REFERENCE TO RELATED APPLICATIONS

This application is related to U.S. provisional application Ser. Nos. 60/458,663, filed 28 Mar. 2003; 60/442,344, filed 23 Jan. 2003; and 60/441,440, filed 17 Jan. 2003, each of which is incorporated herein by reference.

### BACKGROUND OF THE INVENTION

“Cancer” generally refers to one of a group of more than 100 diseases caused by the uncontrolled, abnormal growth of cells that can spread to adjoining tissues or other parts of the body. Cancer cells can form a solid tumor, in which the cancer cells are massed together, or exist as dispersed cells, as in leukemia. Normal cells divide (reproduce) until maturation is attained and then only as necessary for replacement of damaged or dead cells. Cancer cells are often referred to as “malignant”, because they divide endlessly, eventually crowding out nearby cells and spreading to other parts of the body. The tendency of cancer cells to spread from one organ to another or from one part of the body to another distinguishes them from benign tumor cells, which overgrow but do not spread to other organs or parts of the body. Malignant cancer cells eventually metastasize and spread to other parts of the body via the bloodstream or lymphatic system, where they can multiply and form new tumors. This sort of tumor progression makes cancer a deadly disease. Although there have been great improvements in the diagnosis and treatment of cancer, many people die from cancer each year, and their deaths are typically due to metastases and cancers that are resistant to conventional therapies.

Most drug-mediated cancer therapies rely on poisons, called cytotoxic agents, selective for dividing cells. These drugs are effective, because cancer cells generally divide more frequently than normal cells. However, such drugs almost inevitably do not kill all of the cancer cells in the patient. One reason is that cancer cells can acquire mutations that confer drug resistance. Another is that not all cancer cells divide more frequently than normal cells, and slowly-dividing cancer cells can be as, or even more, insensitive to such poisons as normal cells. Some cancer cells divide slowly, because they reside in a poorly vascularized, solid tumor and are unable to generate the energy required for cell division. As a tumor grows, it requires a blood supply and, consequently, growth of new vasculature. The new vasculature that supports tumor growth is often disordered, leaving significant regions of the tumor under-vascularized and even the vascularized regions subject to intermittent blockage. These under-vascularized and blocked regions of the tumor become hypoxic—they have a lower oxygen concentration than the corresponding normal tissue, and the cells in them exhibit slower rates of division. Thus, the median oxygen concentration of only ten percent of solid tumors falls in the normal range of 40-60 mm Hg, and fifty percent of solid tumors exhibit median oxygen concentrations of less than 10 mm Hg.

In addition to rendering cytotoxic agents that target rapidly dividing cells less effective, the hypoxic environment of the tumor can lead to failures in therapy in other ways. First, oxygen is required for the therapeutic action of some cancer drug and radiation therapies. Second, cancer drugs typically reach a tumor via the bloodstream, and poor vascularization leads to poor distribution of cancer drugs to the hypoxic regions of a tumor. For all of these reasons, the hypoxic areas of the tumor represent a significant source of cancer cells resistant to therapy. Not surprisingly, then, low tumor oxygen levels are associated with a poor response to therapy, increased metastases, and poor survival.

Cancer cells require energy to support their rapid rates of cell division, and even the more slowly dividing cancer cells in the hypoxic regions of tumors require energy to survive (and the lack of oxygen deprives them of energy generation via the Krebs cycle, which requires oxygen). Not surprisingly, then, many cancer cells exhibit, relative to normal cells, increased glucose transport and glycolysis, because energy can be generated by glycolysis in the absence of oxygen. Moreover, increased uptake of glucose is one of the most common signs of a highly malignant tumor. Thus, the reference Dickens, 1943, Cancer Research 3:73, reported that “the typical intact cancer cell exhibits an unusual ability to utilize glucose by the process of anaerobic glycolysis through lactate”. While there has been interest in developing drugs that target anaerobic glycolysis in cancer cells, the FDA has not approved any therapy in which a drug that inhibits anaerobic glycolysis is employed.

Lonidamine (LND), also known as 1-(2,4-dichlorobenzyl)-1-H-indazole-3-carboxylic acid, is an anti-cancer drug approved for single agent use in certain countries in Europe for the treatment of lung, breast, prostate, and brain cancer. LND has not been approved by the FDA for anti-cancer use in the United States. The mechanism of action of LND may involve interference with the energy metabolism of neoplastic cells by disruption of the mitochondrial membrane and by inhibition of hexokinase. LND also has anti-spermatogenic activity and has been shown to inhibit germ cell respiration. LND has been studied for use in the treatment of advanced breast cancer. Mansi et al., September 1991, Br J Cancer 64(3):593-7, reports a phase II study in which LND was administered in a daily divided oral dose of 600 mg. Of the 28 patients evaluable for response, three (11%) achieved a partial response (4-24+ months); three (11%) a minor response; two had stable disease (greater than 3 months); and 20 progressed. The investigators concluded that lonidamine appeared to be active against advanced breast cancer.

Combination studies of LND in advanced breast cancer followed this report, particularly studies in combination with epirubicin or doxorubicin. For examples, see laffaioli et al., September 1995, Breast Cancer Res Treat 35(3):243-8 (epirubicin, LND, and alpha 2b interferon); Gardin et al., January 1996, Eur J Cancer 32A(1):176-7 (LND, epirubicin, and cyclophosphamide); Dogliotti et al., April 1996, J Clin Oncol 14(4):1165-72 (LND and epirubicin); Gebbia et al., November 1997, Anticancer Drugs 8(10):943-8 (cisplatin, epirubicin, and LND); Amadori et al., June 1998, Breast Cancer Res Treat 49(3):209-17 (LND and doxorubicin); Dogliotti et al., 1998, Cancer Chemother Pharmacol 41(4):333-8 (cisplatin, epirubicin, and LND); Nistico et al., August 1999, Breast Cancer Res Treat 56(3):233-7 (epirubicin and LND); and Pacini et al., May 2000, Eur J Cancer 36(8):966-75 (FEC (5-fluorouracil, epidoxorubicin and cyclophosphamide) versus EM (epidoxorubicin and mitomycin-C) with or without LND). Berruti et al., 15 Oct. 2002, J Clin Oncol 20(20):4150-9, reported that, in a phase III study with a factorial design, time to progression in metastatic breast cancer patients treated with epirubicin was not improved by the addition of either cisplatin or LND (see also Berruti et al., July-August 1997, Anticancer Res 17(4A):2763-8).

Lonidamine has also been studied in lung cancer (see the reference Joss et al., September 1984, Cancer Treat Rev 11(3):205-36) in combination with radiation or anti-cancer agents. For examples, see Privitera et al., December 1987, Radiother Oncol 10(4):285-90 (LND and radiotherapy); Gallo-Curcio et al., December 1988, Semin Oncol 15(6 Suppl 7):26-31 (chemotherapy or radiation therapy plus and minus LND); Giaccone et al, 28 Feb. 1989, Tumori 75(1):43-6 (LND versus polychemotherapy); lanniello et al., 1 Jul. 1996, Cancer 78(1):63-9 (cisplatin, epirubicin, and vindesine with or without LND); Gridelli et al., March-April 1997, Anticancer Res. 17(2B):1277-9 (VM-26 plus LND); Comella et al., May 1999, J Clin Oncol 17(5):1526-34 (cisplatin, gemcitabine, and vinorelbine with or without LND); DeMarinis et al., May-June 1999, Tumori 85(3):177-82 (vindesine and LND); and Portalone et al., July-August 1999, Tumori 85 (4):239-42 (cisplatin, epidoxorubicin, vindesine, and LND).

Lonidamine has been studied as a treatment for other cancers (see Robustelli et al., April 1991, Semin. Oncol. 18(2 Suppl 4):18-22; and Pacilio et al., 1984, Oncology 41 Suppl 1:108-12), including B-cell neoplasms (see Robins et al., April 1990, Int J Radiat Oncol Biol Phys. 18(4):909-20); advanced colorectal cancer (see Passalacqua et al., June 30 1989, Tumori 75(3):277-9, and Zaniboni et al., November-December 1995, Tumori 81(6):435-7); advanced gastric carcinoma (see Barone et al., 15 Apr. 1998, Cancer 82(8):1460-7); malignant glioma (see Carapella et al., May 1989, J Neurooncol 7(1):103-8, and July-December 1990, J Neurosurg Sci. 34(34):261-4); metastatic cancers (see Weinerman, 1990, Cancer Invest. 8(5):505-8; DeAngelis et al., September 1989, J Neurooncol 7(3):241-7; U.S. Pat. No. 5,260,327; and Weinerman et al., June 1986, Cancer Treat Rep 70(6):751-4); advanced ovarian cancer (see Bottalico et al., November-December 1996, Anticancer Res 16(6B):3865-9; DeLena et al., October 1997, J Clin Oncol 15(10):3208-13, and DeLena et al., February 2001, Eur J Cancer 37(3):364-8); and recurrent papillary carcinomas of the urinary bladder (see Giannotti et al., 1984, Oncology 41 Suppl 1:104-7).

Despite the numerous studies conducted, lonidamine is still not approved for use in the treatment of cancer in the United States, Asia, and most countries in Europe. There remains a need for new methods of treating cancer using lonidamine. The present invention provides such methods for treatment of cancer using lonidamine or a lonidamine analog, alone or in combination with other anti-cancer agents and therapies.

### SUMMARY OF THE INVENTION

The present invention provides methods and compositions for treating cancer and other hyperproliferative disease conditions with lonidamine and lonidamine analogs alone and in combination with other anti-cancer agents and therapies, including radiation and surgery.

In a first aspect, the present invention provides a method of treating or preventing cancer, which method comprises administering to a mammal a therapeutically effective dose of lonidamine or a lonidamine analog. In one embodiment, the method comprises administering to a mammal a therapeutically effective dose of lonidamine or a lonidamine analog in combination with another anti-cancer agent. In one embodiment, the method comprises administering to a mammal a therapeutically effective dose of lonidamine or a lonidamine analog in combination with surgery and, optionally, administration of another anti-cancer agent. In one embodiment, the method comprises administering to a mammal a therapeutically effective dose of lonidamine or a lonidamine analog in combination with radiation therapy and, optionally, administration of another anti-cancer agent.

In a preferred embodiment, the present invention provides methods for treating cancer in a patient, said method comprising administering to the patient an effective amount of lonidamine or a lonidamine analog and an effective amount of one or more additional chemotherapeutic agent(s). In one embodiment, the additional chemotherapeutic agents are selected from the group consisting of gemcitabine, a taxane (including but not limited to paclitaxel and docetaxel), vinorelbine, and 2-deoxy-D-glucose. In one embodiment, the cancer is non-small cell lung cancer. In another embodiment, the cancer is a multi-drug resistant cancer or a cancer that is otherwise refractory to treatment.

In a second aspect, the present invention provides an oral formulation of lonidamine or a lonidamine analog and an additional chemotherapeutic agent. In one embodiment, the additional chemotherapeutic agent is 5-fluorouracil or a pro-drug form thereof. In another embodiment, the additional chemotherapeutic agent is 2-deoxy-D-glucose.

In a third aspect, the present invention provides a method for treating a hyperproliferative disease condition in a human or other mammal, said method comprising administering to the human or other mammal a therapeutically effective dose of lonidamine or a lonidamine analog. In one embodiment, the hyperproliferative disease condition is selected from the group consisting of diseases of inflammation and autoimmune disease, including but not limited to arthritis and psoriasis.

In a fourth aspect, the present invention provides a sustained release formulation of lonidamine or a lonidamine analog. In one embodiment, the formulation is orally administered and permits once-a-day dosing of a therapeutically effective dose of lonidamine or a lonidamine analog.

In a fifth aspect, the present invention provides methods for treating cancer that involve a preliminary assessment of the cancer patient to determine the degree of susceptibility of the patient's cancer to lonidamine-mediated drug therapy.

In related aspects, the invention provides the use of lonidamine or a lonidamine analog in the manufacture of a medicament for the treatment of cancer. In one embodiment, the medicament is administered daily for at least three days in a one week period. In one embodiment, the medicament is administered orally. In one embodiment, the medicament is a tablet or capsule. In one embodiment, the cancer is a cancer other than breast cancer, cervical cancer, lung cancer, or prostate cancer. In one embodiment, the cancer is breast cancer, cervical cancer, lung cancer, colon (colorectal) cancer, or prostate cancer. In one embodiment, the cancer is a multi-drug resistant cancer or a cancer that is otherwise refractory to treatment. In one embodiment, the cancer is a taxane-resistant cancer, and the method comprises administration of a taxane and lonidamine or a lonidamine analog at a therapeutically effective dose. In another related aspect, the invention provides the use of lonidamine or a lonidamine analog in the manufacture of a medicament for use in combination with another anticancer agent for the treatment of cancer. In another related aspect, the invention provides the use of lonidamine or a lonidamine analog in the manufacture of a medicament for use in combination with another metabolic inhibitor for the treatment of cancer.

These and other aspects and embodiments of the invention are described in detail in the following description and example.

### BRIEF DESCRIPTION OF THE FIGURES

FIG. 1 shows the expression of HIF-1alpha in LNCaP cells under normoxic and hypoxic conditions and in the presence and absence of lonidamine. FIG. 1A shows an assay using a nuclear extract. FIGS. 1B and 1C show an assay using a whole cell extract.

FIG. 2 shows shows the expression of HIF-1alpha in PC-3 cells under normoxic and hypoxic conditions and in the presence and absence of lonidamine. FIGS. 2A and 2C show an assay using a nuclear extract. FIG. 2B shows an assay using a whole cell extract.

FIG. 3 shows the effect of 0-600 microM lonidamine on expression of HIF-1alpha and other proteins as determined in whole cell extracts from LNCaP cells cultured under hypoxic conditions.

FIG. 4 shows the effect of 0-600 microM lonidamine on expression of HIF-1alpha and other proteins as determined in nuclear extracts from LNCaP cells cultured under hypoxic conditions.

### DETAILED DESCRIPTION OF THE INVENTION

The present invention provides methods of treating cancer by administering a therapeutically effective dose of lonidamine or a lonidamine analog, alone or in combination with other anti-cancer therapies, including surgical resection, radiation therapy, and drug therapy. The present invention provides also provides pharmaceutical compositions useful in the treatment of cancer. In a preferred embodiment, administration of a combination of lonidamine or a lonidamine analog and another chemotherapeutic agent as described herein results in a tumor cell-killing effect that is at least additive and, with certain anti-cancer agents, synergistic with the administration of either agent alone.

Lonidamine is the generic name for 1-(2,4-dichlorobenzyl)-1H-indazole-3-carboxylic acid, and has also been referred to in the medical literature as 1-[(2,4-dichlorophenyl)-methyl]-1H-indazole-3-carboxylic acid, AF1890, diclondazolic acid (DICA), and Doridamina™ (ACRAF; Aziende Chimiche Riunite Angelini Francesco). Lonidamine was first identified as an anti-spermatogenic agent, and subsequently approved for the treatment of a limited number of cancers in only a few countries in Europe. See Silvestrini, 1981, “Basic and Applied Research n the Study of Indazole Carboxylic Acids” Chemotherapy 27:9-20. The mechanisms of action of lonidamine in spermatogenesis and cancer may not be completely understood. However, it has been suggested that lonidamine's anticancer properties result at least in part from a lonidamine-mediated disruption of the mitochondrial membrane, resulting in reduced activity of mitochondrially-bound hexokinase and interference with ATP production by the glycolytic pathway and oxidative phosphorylation. See, Floridi et al., 1981, “Effect of lonidamine on the energy metabolism of Ehrlich ascites tumor cells” Cancer Res. 41:4661-6, Fanciulli et al., 1996, “Effect of the antitumor drug lonidamine on glucose metabolism of adriamycin-sensitive and -resistant human breast cancer cells” Oncology Research 3:111-120, and references numbered 15-22 therein; and Gatto et al., 2002, “Recent studies on lonidamine, the lead compound of the antispermatogenic indazol-carboxylic acids” Contraception 65:277-78. Also see Kaplan, 2000 “Correspondence re: M. Fanciulli et al., Energy metabolism of human LoVo colon carcinoma cells: correlation to drug resistance and influence of lonidamine.” Clin Cancer Res. 6:4166-7.

To aid in the appreciation of the invention, this description is divided into the following topics: (i) Definitions; (ii) Structure and synthesis of lonidamine analogs; (iii) Therapeutically effective administration of lonidamine and lonidamine analogs for the treatment of cancer; (iv) Co-administration with other anti-cancer agents and other metabolic inhibitors; (v) Treatment of particular cancers; (vi) Formulations; and (vii) Theranostics. The description below is organized into sections for convenience only, and disclosure found in any organizational section is applicable to any aspect of the invention.

(I) Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of cancer, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of cancer refers to a symptom, sign, anatomical state, physiological state, or report characteristic of a subject with cancer.

As used herein, a “therapeutically effective amount” of a drug is an amount of a drug that, when administered to a subject with cancer, will have the intended therapeutic effect, e.g., alleviation, amelioration, palliation or elimination of one or more manifestations of cancer in the subject. The full therapeutic effect does not necessarily occur by administration of one dose, and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

As used herein, “TID” and “QD” have their ordinary meanings of “three times a day” and “daily,” respectively.

As used herein “patient” or “subject” typically refers to a human, but more generally refers to a mammal. Those of skill in the art will appreciate that the methods and compositions of the invention can be used to treat cancer in any mammal, including non-human primates and experimental models of human cancers. In one embodiment of the invention the patient is a human patient.

As used herein, “alkyl” refers to a monovalent alkane (hydrocarbon) derived radical containing from 1 to 15 carbon atoms. It may be straight, branched or cyclic and may be unsubstituted or substituted with substituent groups including but not limited to hydroxyl, halide, alkoxyl, and nitrile. Alkoxy groups that can be used include but are not limited to methoxy. Illustrative straight or branched alkyl groups include methyl, ethyl, propyl, isopropyl, butyl and t-butyl.

As used herein, “aryl” refers to moieties that include one or more monocyclic or fused ring aromatic systems. Such moieties include any moiety that includes one or more monocyclic or bicyclic fused ring aromatic systems, including but not limited to phenyl and naphthyl. Aryl groups may be unsubstituted or substituted with substituent groups as listed for the particular substituted aryl.

As used herein, “heteroaryl” refers to monocyclic aromatic groups having 5 or 6 ring atoms, or fused ring bicyclic aromatic groups having 8 to 10 atoms, in which the ring atoms are C, O, S, SO, SO₂, or N and at least one of the ring atoms is a heteroatom, i.e., O, S, SO, SO₂, or N. Heteroaryl groups may be unsubstituted or substituted with substituent groups as listed for the particular substituted heteroaryl. Examples of monocyclic aromatic heteroaryl groups include but are not limited to pyridyl. Examples of bicyclic fused ring heteroaryl groups include but are not limited to indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl. Unless indicated otherwise, the arrangement of the hetero atoms within the ring may be any arrangement allowed by the bonding characteristics of the constituent ring atoms.

As used herein, the terms “heterocycloalkyl” and “heterocyclyl” refer to a monocyclic or fused ring multicyclic cycloalkyl group at least a portion of which is not aromatic and in which one or more of the carbon atoms in the ring system is replaced by a heteroatom selected from O, S, SO, SO₂, or N. Examples of heterocyclyl groups include but are not limited to piperidinyl, morpholinyl, pyrrolidinyl, tetrahydrofuranyl, tetrahydroimidazo [4,5-c] pyridinyl, imidazolinyl, piperazinyl, pyrrolidine-2-onyl, and piperidin-2-onyl.

As used herein, “cycloalkyl” refer to a monocyclic or fused ring multicyclic group at least a portion of which is not aromatic and in which the ring atoms are carbon.

As used herein “heterocycloalkenyl” refers to a monocyclic or fused ring multicyclic group in which one or more of the carbon ring atoms is replaced by a hetero atom, the ring system is at least partially not aromatic, and the ring system includes at least one carbon-carbon double bond.

ii) Structure and Synthesis of Lonidamine Analogs

Lonidamine and lonidamine analogs and derivatives can be prepared using by well known synthetic methods. The structures of lonidamine (compound I; R═Cl) and the lonidamine analogs tolnidamine (compound I, R═CH₃), AF-2364 (compound II) and AF-2785 (compounds III) are shown below.

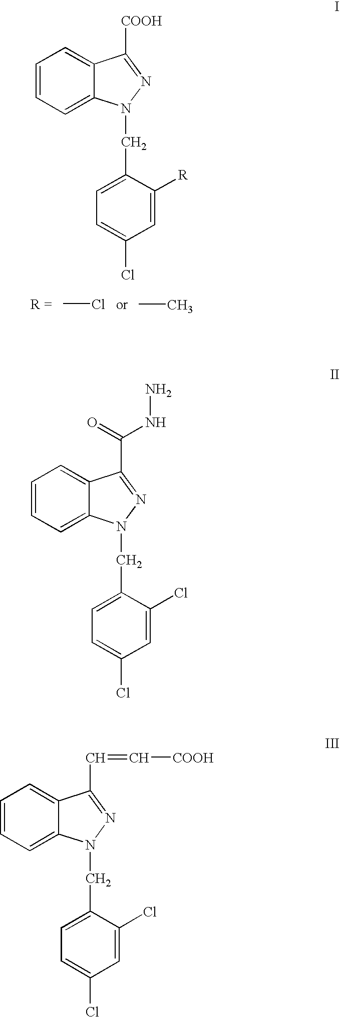

Synthesis of lonidamine is described in U.S. Pat. No. 3,895,026 and Germany Patent No. 2,310,031. Synthesis of exemplary lonidamine analogs, including tolnidamine (TND), is described in the art (see, e.g., Corsi et al., 1976, “1-Halobenzyl-1H-Indazole-3-Carboxylic Acids. A New Class of Antispermatogenic Agents”, Journal of Medicinal Chemistry 19:778-83, and Cheng et al., 2001, “Two new mate contraceptives exert their effects by depleting germ cells prematurely from the testis” Biol Reprod. 65:449-61) (see also, e.g., Corsi et al., 1976, “1-Halobenzyl-1H-Indazole-3-Carboxylic Acids. A New Class of Antispermatogenic Agents”, Journal of Medicinal Chemistry 19:778-83; Cheng et al., 2001, “Two new male contraceptives exert their effects by depleting germ cells prematurely from the testis” Biol Reprod. 65:449-61; Silvestrini, 1981, “Basic and Applied Research in the Study of Indazole Carboxylic Acids” Chemotherapy 27:9-20; Lobl et a/., 1981, “37 Effects of Lonidamine (AF 1890) and its analogues on follicle-stimulating hormone, luteinizing hormone, testosterone and rat androgen binding protein concentrations in the rat and rhesus monkey”” Chemotherapy 27:61-76; and U.S. Pat. Nos. 3,895,026 and 6,001,865). It will be appreciated, of course, that lonidamine analogs useful in the practice of the invention are not limited to those for which specific structures are provided in this disclosure or the cited references, and that the compounds described above are provided for illustration and not to limit the present invention. It also will be clear that lonidamine analogs useful in the methods of the present invention are not limited to those now described herein or elsewhere in the pharmaceutical and patent literature; the ordinarily skilled practitioner guided by the present disclosure can synthesize novel analogs suitable for use according to the present invention using routine methods in medicinal chemistry.

In certain embodiments, lonidamine or a lonidamine analog is provided in the form of a pharmaceutically acceptable salt. Pharmaceutically acceptable salts include addition salts with acids, as well as the salts with bases. Salts with bases are, for example; alkali metal or alkaline earth metal salts, such as sodium, potassium, calcium or magnesium salts, or ammonium salts, such as those with ammonia or suitable organic amines, e.g. diethylamine, di-(2-hydroxyethyl)-amine or tri-(2-hydroxyethyl)-amine. Suitable acids for the formation of acid addition salts are, for example, mineral acids, such as hydrochloric, hydrobromic, sulphuric or phosphoric acid, or organic acids, such as organic sulphonic acids, for example, benzenesulphonic, 4-toluenesulphonic or methanesulphonic acid, and organic carboxylic acids, such as acetic, lactic, palmitic, stearic, malic, maleic, fumaric, tartaric, ascorbic or citric acid.

Administration of ester, amide and prodrug derivatives of lonidamine and analogs is also contemplated in the practice of the present invention (see, e.g., U.S. Pat. No. 6,146,658, for general information regarding preparation of such derivatives from a compound of interest) as is administration of polymorphic forms, enantiomeric forms, tautomeric forms, solvates, hydrates, and the like.

A variety of compounds structurally related to lonidamine are useful for the treatment of cancer. Useful compounds are generally structurally similar to, are bioisosteres of, or are pharmacophores of lonidamine, as described below, and have biological activity(s) similar to those of lonidamine, as also discussed below. Such compounds can be referred as “bioactive lonidamine analogs,” “Ionidamine analogs,” or, in some cases, simply, “analogs.”

Structural characteristics of lonidamine analogs. Based, in part, on the structure of lonidamine and related compounds known to have pharmaceutical activities similar to that of lonidamine, certain lonidamine analogs, including novel analogs provided by the present invention, suitable for use in treatment of cancer as described herein are described by the formula, 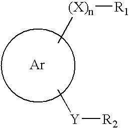
where R₁, R₂, X, Y, n and 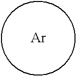
are defined below.

R₁ represents —COOH or a derivative or bioisostere of the —COOH group. R₁ is usually selected from an acid group of formula —COOH; an amide of formula —CONR₃R₄, where R₃ and R₄ may be independently alkyl or hydrogen, with hydrogen preferred; a hydrazide of formula —CONHNR₆R₇, where R₆ and R₇ are usually —H or —CH₃; a substituted ester of formula —COOR₅, with R₅ being a residue easily hydrolyzed in the subject after administration and generally a straight chain or branched chain alkyl group substituted with one or more hydroxyl groups, more usually a straight chain or branched chain methyl, ethyl, or propyl group substituted with one or more hydroxyl groups, more usually still an ethyl group substituted with one hydroxyl group or a straight chain or branched chain propyl group substituted with two hydroxyl groups, and most usually —CH₂CH₂OH, —CH₂CH(OH)CH₂OH, or —CH₂(CH₂OH)₂. R₁ may also be the carboxylate anion of formula —COO⁻, in which case the lonidamine or lonidamine analog will be associated with a counter ion, Z⁺, where Z⁺ is a pharmaceutically acceptable cation.

R₂ represents a substituted or unsubstituted aryl or heteroaryl group. Usually, R₂ is a substituted aryl group; more usually, a substituted phenyl group; more usually still, a phenyl group substituted by one, two, or three substituents independently selected from halo and alkyl substituents, particularly —Cl, —Br, —I, CF₃ and —CH₃ substituents. When R₂ is a substituted phenyl group, R₂ is usually —Cl, —Br, —I, CF₃ or —CH₃, monosubstituted phenyl, substituted at the 2, 3, or 4 position; dichloro, dibromo, dimethyl, or chloro and methyl disubstituted phenyl, substituted at the 2 and 3 or 2 and 4 positions; or 2, 4, 5 trichlophenyl. When R₂ is a substituted phenyl group, R₂ is more usually 2,4-dichlorophenyl or 4-chloro-2-methylphenyl.

X represents a straight chain or branched chain, saturated or unsaturated hydrocarbon linkage group. When X is a saturated hydrocarbon linkage group, X is usually a straight chain linkage group and usually X has the formula —(CH₂)ₚ—, with p equal to 1, 2, or 3. When X is a saturated hydrocarbon linkage group, X is most usually a methylene group, —(CH₂)—. When X is an unsaturated hydrocarbon linkage group, X is usually a straight chain linkage group, most usually —(CH═CH)—.

Y represents a moiety of formula —CHR₇—, where R₇ is hydrogen or a straight chain or branched chain alkyl group, more usually R₇ is hydrogen or or a straight chain alkyl group, more usually still R₇ is hydrogen, methyl, ethyl, or n-propyl, more usually still R₇ is hydrogen or methyl, and most usually R₇ is hydrogen (i.e., Y is most usually —CH₂—).

n is zero or, most usually, one. 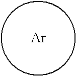
is a core ring system that may generally be an aryl, heteroaryl, cycloalkyl or heterocyclyl ring system. The Ar core ring system usually includes 2 fused rings. The fused rings may generally be 4-, 5-, 6-, 7-, or 8-membered rings, more usually 5- or 6-membered rings. The core ring system is most usually fused 5- and 6-membered rings. The fused ring atoms may generally be any atom, usually carbon or hetero atoms, more usually carbon and nitrogen group atoms, and more usually still carbon and nitrogen. The number of carbon atoms in the core ring system is usually 7. The core ring system usually contains 2 hetero atoms, where the preferred hetero atom is nitrogen. Generally, one or more of the fused rings may be aromatic. When the core ring system is fused 5- and 6-membered rings, the core ring system is usually aromatic over both fused rings. The fused 5- and 6-membered ring system is most usually an indazole.

More particularly, lonidamine analogs for use according to the methods of the invention, and certain of the novel analogs provided by the invention, include analogs of the formula 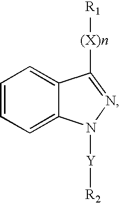
where R₁, R₂, X, Y, and n are generally as above or, in a preferred version,
R₂ is —Cl, —Br, —I, or —CH₃, monosubstituted phenyl, substituted at the 2, 3, or 4 position; dichloro, dibromo, dimethyl, or chloro and methyl disubstituted phenyl, substituted at the 2 and 3 or 2 and 4 positions; or 2, 4, 5 trichlophenyl;
Y is —(CH₂)—; and
n is zero, and R₁ is —COOH, —CONH₂, —CONHNH₂, —CONHN(CH₃)₂, —CH₂CH₂OH, —CH₂CH(OH)CH₂OH, or CH₂(CH₂OH)₂; or
n is one, R₁ is —COOH, and X is —CH═CH—.

In one embodiment, the lonidamine analog is a 1,3-substituted-indazole, such as a 1-halobenzyl-1H-indazole. In another embodiment, the lonidamine analog is a 3-substituted 1-benzyl-1H-indazole. In another embodiment, the lonidamine analog is a 1-substituted-indazole-3-carboxylic acid, such as a 1-halobenzyl-1H-indazole-3-carboxylic acid.

Bioisosteres. In addition, lonidamine analogs that may be used in the treatment methods of the invention include bioisosteres and pharmacophores of lonidamine and analogs described herein. Bioisosterism is a well-known tool for predicting the biological activity of compounds, based upon the premise that compounds with similar size, shape, and electron density can have similar biological activity. To form a bioisostere of a given molecule, one replaces one or more atoms or groups with known bioisosteric replacements for that atom or group. Known biolsosteric replacements include, for example, the interchangeability of —F, —OH, —NH₂, —Cl, and —CH₃; the interchangeability of —Br and -i-C₃H₇; the interchangeability of —I and -i-C₄H₉; the interchangeability of —O—, —S—, —NH—, —CH₂, and —Se—; the interchangeability of —N═, —CH═, and —P═ (in cyclic or noncyclic moieties); the interchangeability of phenyl and pyridyl groups; the interchangeability of —C═C— and —S— (for example, benzene and thiophene); the interchangeability of an aromatic nitrogen (R₁—N(R₃)—R₂) for an unsaturated carbon (R₁—C(═R₃)—R₂); and the interchangeability of —CO—, —SO—, and —SO₂—. These examples are not limiting on the range of bioisosteric equivalents and one of skill in the art will be able to identify other bioisosteric replacements known in the art. See, e.g., Patani and LaVoie, 1996; and Burger, 1991.

Pharmacophores. In addition to the lonidamine analogs described herein, lonidamine analogs that may be used in the methods of the invention can generally be any pharmacophore of lonidamine and the lonidamine analogs described above. Often a reasonable quantitative prediction of the binding ability of a known molecule can be made based on the spatial arrangement of a small number of atoms or functional groups in the molecule. Such an arrangement is called a pharmacophore, and once the pharmacophore or pharmacophores in a molecule have been identified, this information can be used to identify other molecules containing the same or similar pharmacophores. Such methods are well known to persons of ordinary skill in the art of medicinal chemistry, and as the structural information described in this application identifies the pharmacophore of lonidamine and the lonidamine analogs relevant to treatment of cancer, those of skill in the art can identify other LND analogs that comprise the pharmacophore and so are useful in treating cancer. An example of programs available to perform pharmacophore—related searches is the program 3D Pharmacophore search from the Chemical Computing Group (see http://www.chemcomp.com/fdept/prodinfo.htm).

A lonidamine analog of particular interest is tolnidamine (1-(4-chloro-2-methylbenzyl)-1H-indazole-3-carboxylic acid, AF 1923); Ansari et al., 1998, “Long-term sequelae of tolnidamine on male reproduction and general body metabolism in rabbits” Contraception 57:271-79; Corsi et al., 1979. Tolnidamine (TND) differs from lonidamine by the presence of a methyl substituent, rather than a chlorine substituent, in position 2 of the benzyl group. Other analogs of lonidamine with biological activity have been described in the following publications: U.S. Pat. No. 3,895,026 entitled “Substituted 1-Benzyl-1H-Indazole-3-Carboxylic Acids and Derivatives Thereof;” Corsi et al., 1976, “1-Halobenzyl-1H-Indazole-3-Carboxylic Acids. A New Class of Antispermatogenic Agents,” Journal of Medicinal Chemistry 19:778-83; Silvestrini, 1981, “Basic and Applied Research in the Study of Indazole Carboxylic Acids,” Chemotherapy 27:9-20; Lobl et al., 1981, “Effects of Lonidamine (AF 1890) and its analogues on follicle-stimulating hormone, luteinizing hormone, testosterone and rat androgen binding protein concentrations in the rat and rhesus monkey,” Chemotherapy 27:61-76; U.S. Pat. No. 6,001,865 entitled “3-Substituted 1-Benzyl-1H-Indazole Derivatives As Antifertility Agents”; and Cheng et al., 2001, “Two new male contraceptives exert their effects by depleting germ cells prematurely from the testis,” Biol Reprod. 65:449-61, which describe AF-2364 and AF-2785 and other compounds.

The activity of a lonidamine analog of interest in any of the aforementioned assays can be compared with that of lonidamine to provide guidance concerning dosage schedules for the compound, and other information. Generally, lonidamine analogs with greater biological activity per mg than lonidamine are of special interest.

(iii) Therapeutically Effective Administration of Lonidamine and Lonidamine Analogs for the Treatment of Cancer

In accordance with the methods of the invention, lonidamine (LND) or a lonidamine analog is administered to a patient to treat cancer. In one embodiment, LND or the lonidamine analog is administered daily in an amount (total daily dose) ranging from about 100 mg to about 1 g. In another embodiment, LND or the lonidamine analog is administered daily in an amount ranging from about 300 to about 750 mg. In another embodiment, LND or the lonidamine analog is administered daily in an amount of about 450 to 500 mg tid. In another embodiment, a sustained release formulation of LND or the lonidamine analog provided by the invention is administered once per day in an amount within the ranges specified above.

A variety of routes and dosage schedules are appropriate for administration of lonidamine and lonidamine analogs according to the invention.

A preferred mode of delivery of lonidamine and lonidamine analogs to a patient is oral delivery. Preferred dosage forms for oral administration are pills, tablets, capsules, caplets, and the like, especially as formulated for sustained release. Other suitable forms for oral administration include troches, elixirs, suspensions, syrups, wafers, lozenges, and the like. Other modes of administration are also contemplated, including parenteral, inhalation spray, transdermal, rectal, intraprostetic injection (e.g., of lonidamine-containing microparticles) and other routes. Ionidamine and lonidamine analogs may be formulated in suitable dosage unit formulations containing conventional non-toxic pharmaceutically acceptable carriers, adjuvants and vehicles appropriate for each route of administration. In one embodiment, the dosage form is the 150 mg unit dosage form marketed in Italy under the tradename Doridamina (ACRAF).

The dose, schedule and duration of administration of lonidamine and lonidamine analogs will depend on a variety of factors, including the age, weight and health of the subject, the type of cancer being treated, the subject's medical history, co-treatments, therapeutic goal (e.g., therapy or prophylaxis), the mode of administration of the drug, the formulation used, patient response to the drug, and the like. For illustration rather than limitation, two general categories of dosing for administration of lonidamine and lonidamine analogs can be described: high dosing low dosing. For reference, the standard lonidamine dose used for the treatment of the specific types of cancer for which lonidamine has been approved in a few countries in Europe is 150 mg po TID for about thirty days ( an example of “low dosing” as described herein).

It will be appreciated that these dosing schedules are for illustration and not limitation, and that a dosing schedule may change during a course of therapy based on, for example, a patient's response to the therapy or the use of a lonidamine analog that has an activity/dose profile significantly different from that of lonidamine.

As noted, the daily dosages recommended herein may be divided for, for example, two-, three- or four-times per day administration. In one embodiment, the drug is formulated for administration once-per day. In one embodiment, the drug is formulated for administration less frequently than once per day. In another embodiment, a modified-release form of the drug is used.

Low dosing. In one embodiment, cancer is treated in accordance with the methods of the invention by administering lonidamine or a lonidamine analog to a cancer patient at a dose defined herein as a “low dose”. Exemplary low doses include, without limitation, doses greater than 300 and less than 500 mg/day, such as doses in the range >300-400 or 400<500 (e.g., 450 mg/day). The daily dosages may be divided, for example, for two-, three- or four-times per day administration. In an embodiment, the drug is formulated for administration once-per day or less frequently than once per day. In one embodiment, a modified-release form of the drug is used. Alternatively, this low dose can be administered on a one-time, once-a-week, once every two weeks, or once-a-month basis (e.g., 300-500 mg/administration) or by other schedules to be determined by the administering physician. In one embodiment, the daily dosage is 150 mg of lonidamine or a lonidamine analog taken three times a day.

A low dose schedule may be used for therapy or prophylaxis. In one embodiment, a low dose is administered in combination with, or following, a surgical and/or hyperfractionated radiation treatment for cancer.

Administration of low doses of lonidamine can be daily, every other day, five days on, two days off, and other schedules determined by the administering physician.

An advantage of the low dose schedules of the invention is that this dose may be continued to be administered for weeks to months while limiting or eliminating the unwanted, albeit usually mild, side effects reported for higher doses of lonidamine (principally myalgia and testicular pain).

A low dose schedule can be used for therapy or prophylaxis (prevention of recurrence). In one embodiment, a low dose form is used for a maintenance dose after a higher initial, priming or loading dose.

High dosing, In another embodiment, cancer is treated in accordance with the methods of the invention by administering to a cancer patient a higher dose of lonidamine or a lonidamine analog (usually for a shorter period of time than for low doses). Exemplary high doses include, without limitation, total daily doses greater than 0.5 g, such as doses in the range 0.5-5 g/day, 0.5-3 g/day, 0.5-1 g/day and 1-3 g/day, or higher doses. The daily dosages may be divided, for example, for two-, three- or four-time per day administration. In one embodiment, the drug is formulated for administration once-per day, or less frequently than once per day. In one embodiment, a modified-release form of the drug is used. Alternatively, a high dose can be administered on a one-time, once-a-week, once every two weeks, or once-a-month basis (e.g., 0.5-5 g/administration) or by other schedules to be determined by the administering physician.

A high dose schedule can be used for therapy or prophylaxis. In one embodiment, a high dose is administered in combination with, or following, a surgical or other non-drug treatment for cancer.

Duration. In therapeutic and prophylactic applications, lonidamine or the lonidamine analog can be administered a single time or many times over periods as long as a month to several months or a year.

(iv) Co-Administration with Other Anti-Cancer Agents and Metabolic Inhibitors

In accordance with the methods of the invention, lonidamine or a lonidamine analog can be co-administered in combination with other anti-cancer agents (“anticancer agent”). Without intending to be bound by any particular mechanism or effect, such co-administration can in some cases provide one or more of several unexpected benefits including:

(i) co-administration of lonidamine or a lonidamine analog and the anticancer agent has a synergistic effect on induction of cancer cell death;

(ii) co-administration provides a better therapeutic result than administration of the anticancer agent alone, e.g., greater alleviation or amelioration of one or more symptoms of the cancer, diminishment of extent of disease, delay or slowing of disease progression, amelioration, palliation or stabilization of the disease state, partial or complete remission, prolonged survival or other beneficial therapeutic results;

(iii) co-administration of lonidamine or a lonidamine analog increases the sensitivity of cancer cells to the anticancer agent, allowing lower doses of the agent to be adminstered to the patient or allowing an agent to be used for treatment of cells otherwise resistant to the agent or otherwise refractory to treatment;

(iv) co-administration of lonidamine or a lonidamine analog and the anticancer agent increases killing of cells in hypoxic regions of tumors that are not efficiently killed by the agent alone.

As used herein, lonidamine or a lonidamine analog is “co-administered” with another anticancer agent (also referred to herein as, “Agent”) when the lonidamine or a lonidamine analog and Agent are administered as part of the same course of therapy. In one embodiment, lonidamine or a lonidamine analog is first administered prior to administration of the Agent, (i.e., the initiation of the other cancer therapy), and treatment with lonidamine or a lonidamine analog is continued throughout the course of administration of the Agent (i.e., the course of the other therapy). In another embodiment, lonidamine or a lonidamine analog is administered after the initiation or completion of the other cancer therapy. In other embodiments, lonidamine or a lonidamine analog is first administered contemporaneously with the initiation of the other cancer therapy. In one embodiment, lonidamine or a lonidamine analog is first administered prior to administration of the Agent, and treatment with lonidamine or a lonidamine analog is continued after the cessation of administration of the Agent. In one embodiment, lonidamine or a lonidamine analog is first administered prior to administration of the Agent, and treatment with lonidamine or a lonidamine analog is continued during part of the period of administration of the Agent.

Anticancer drug therapy today typically involves multiple rounds, or “cycles,” of administration of the anti-cancer agent(s), and typically, more than one Agent is administered. In the context of administering lonidamine or a lonidamine analog, each cycle of administration (as well as a complete set of cycles) can be viewed as administration of a second drug. Thus, lonidamine or a lonidamine analog can be administered in any or all of the multiple cycles of treatment with the other Agent; in general, lonidamine or a lonidamine analog will be given on a daily basis for at least two or more days during each cycle. In one aspect of the invention, lonidamine or a lonidamine analog is co-administered with the Agent according to a schedule repeated at each round. For example, in one conventional therapy, paclitaxel is administered at 135 mg/m² by IV as a 24-hour infusion once every 21 days, e.g., Days 21, 42, 63, and 84 of a course of treatment. In this example, each round of paclitaxel administration can be accompanied by lonidamine or-a lonidamine analog co-administration which is concurrent with the paclitaxel administration (e.g., lonidamine or a lonidamine analog is administered on Days 21, 42, 63, and 84), precedes the paclitaxel administration (e.g., lonidamine or a lonidamine analog is administered on Days 20,41, 62, and 83), and immediately after the paclitaxel administration (e.g., lonidamine or a lonidamine analog is administered on Days 22, 43, 64, and 85; or if administered qod during roughly the same periods, Days 21 and 23; 42 and 44, 63 and 65; and 84 and 86). For convenience, however, and particularly if the Agent is administered by IV infusion, the physician may omit the preceding day dose of lonidamine or a lonidamine analog for the first cycle. Alternatively, lonidamine or a lonidamine analog may be administered continuously throughout multiple cycles of administration of the anticancer Agent (e.g., in the pacitaxal example, daily beginning on or before day 21 and extending until the end of therapy; every other day beginning on or before day 21 and extending until the end of therapy, etc.). It will be understood that the above examples are for illustration only, and not intended to limit the invention in any fashion. Those of skill in the art will also appreciate that in many cases the schedule of co-admistration may differ in the first therapeutic cycle for the convenience of the patient (e.g., no lonidamine or a lonidamine analog administration prior to the first administration of paclitaxel).

In one embodiment, lonidamine or a lonidamine analog is administered with an anti-cancer agent that is more effective when ATP levels in the cancer cell are low. In this embodiment, the therapy of the invention optionally includes an assay or test to measure ATP levels (or a surrogate marker) in the tumor to be treated. Lonidamine acts in part by reducing the ATP available to the cancer cell. Thus, in one aspect of the invention, lonidamine or a lonidamine analog is administered once in an amount effective for reducing ATP levels in the tumor and administered again only after ATP levels begin to rise; thereafter, lonidamine or a lonidamine analog is administered to maintain ATP at a low level in the tumor. Thus, a single dose of lonidamine or a lonidamine analog that reduces ATP in a cancer cell can have a therapeutic effect. The DNA damage induced by radiation therapy and by certain drug therapies, such as treatment with an alkylator, cross-linking agent, or other DNA modifier, requires ATP for repair. Consequently, administration of lonidamine or a lonidamine analog in accordance with the methods of the present invention can improve patient outcomes when conducted concurrently with such therapies. In one embodiment of this method, lonidamine or a lonidamine analog is administered contemporaneously with the administration of the DNA damaging agent, and administration of lonidamine or a lonidamine analog is stopped when the other treatment is stopped or within a few days thereafter.

In a related embodiment, lonidamine or a lonidamine analog is administered in combination with another anti-cancer agent in accordance with the methods of the invention to treat a multipl-drug resistant tumor, and this treatment method can optionally include a step to diagnose whether a tumor is multiply drug resistant. This step can simply be the administration of a drug and the observation that the cancer appears to be resistant to the drug or a diagnostic test for the presence of an RNA, a protein, or an activity associated with multiple drug resistance. Multiple drug resistance can arise from the expression of certain proteins, including P-glycoprotein (PGP), multidrug-resistance protein (MRP), and lung-resistance protein (LRP). PGP causes resistance to anthracyclines (such as doxorubicin, daunorubicin, and epirubicin), mitxantrone, vinca alkaloids (vinblastine, vincristine), etoposide, the taxanes (paclitaxel, docetaxel), and actinomycin D; MRP causes resistance to anthracyclines, vinca alkaloids, and etoposide; and LRP causes resistance to anthracyclines, mitoxantrone, cisplatin (CDDP), and certain alkylating agents. In one embodiment, the therapeutic method of the invention comprises administering to a patient having a multiple-drug-resistant cancer a therapeutically effective regiment of lonidamine or a lonidamine analog together with another anti-cancer agent selected from those agents to which the multiple-drug resistant tumor is otherwise resistant.

In another embodiment, lonidamine or a lonidamine analog is administered with an anti-cancer agent that acts, either directly or indirectly, to inhibit hypoxia-inducible factor 1 alpha (HIF1a) or to inhibit a protein or enzyme, such as a glucose transporter or VEGF, whose expression or activity is increased upon increased HIF1a levels. As described in Example 1, lonidamine itself appears in some tests to have HIF-1alpha inhibitory activity, although the specificity and mechanism of that inhibition have not been determined. HIF1a inhibitors suitable for use in this embodiment of the invention include P13 kinase inhibitors; LY294002; rapamycin; histone deacetylase inhibitors such as [(E)-(1S,4S,10S,21R)-7-[(Z)-ethylidene]-4,21-diisopropyl-2-oxa-1 2,1 3-dithia-5,8,20,23-tetraazabicyclo-[8,7,6]-tricos-16-ene-3,6,9,19,22-pentanone (FR901228, depsipeptide); heat shock protein 90 (Hsp90) inhibitors such as geldanamycin, 17-allylamino-geldanamycin (17-AAG), and other geldanamycin analogs, and radicicol and radicicol derivatives such as KF58333; genistein; indanone; staurosporin; protein kinase-1 (MEK-1) inhibitors such as PD98059 (2′-amino-3′-methoxyflavone); PX-12 (1-methyl propyl 2-imidazolyl disulfide); pleurotin PX-478; quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins; barbituric and thiobarbituric acid analogs; camptothecins; and YC-1, a compound described in Biochem. Pharmacol., 15 Apr. 2001, 61(8):947-954, incorporated herein by reference, and its derivatives. Other agents that can be used in combination with lonidamine or a lonidamine analog to treat cancer include glycolytic inhibitors, inhibitors of NADH/NADPH formation, and ribose-5-phosphate synthesis inhibitors, agents that increase glucose transport (as such agents, which include the taxanes, also increase 2-DG transport), flavopiridol, bryostatin, 7-hydroxystaurosporine, carboxyamide-triazole, KRN5500, spicamycin, rapamycin, non-antibiotic tetracyclines, COL-3, quinocarmycin, DX-52-1, rebeccamycin, bizelesin, dolastin 10, Rhizoxin, cryptophycin, eleutherobin, and analogs and derivatives of the foregoing compounds.

Lonidamine is a metabolic inhibitor and can, in accordance with the methods of the invention, be used in combination with other metabolic inhibitors and optionally with other cytotoxic or anti-cancer agents, to treat cancer. As used herein, a “metabolic inhibitor” is any compound that inhibits glycolysis (for example and without limitation by inhibiting glucose transport or inhibiting hexokinase) and/or mitochondrial function. In one embodiment, 3-bromopyruvate or its 3-halo-pyruvate analogs can also be used in combination with lonidamine or a lonidamine analog to treat cacncer. Other glycolytic inhibitors, mitochondrial function inhibitors, mitochondrial poisons, and hexokinase inhibitors useful in the methods of the present invention are described in PCT patent publication WO 01/82926 and U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference. In one embodiment, the present invention provides a method for treating cancer in a patient by administering to the patient a therapeutically effective dose of lonidamine or a lonidamine analog in combination with another metabolic inhibitor.

A preferred metabolic inhibitor for use in combination with lonidamine or a lonidamine analog is 2-deoxyglucose (2-DG) and analogs (2-DGA) thereof; exemplary dosage schedules are described in co-pending U.S. patent application Ser. No. 10/______ (entitled “Treatment of cancer with 2-deoxyglucose,” attorney docket no. 54492-2000400, filed 9 Jan. 2004 claiming priority to U.S. patent application Ser. No. 60/496,163, filed 18 Aug. 2003, incorporated herein by reference). For example, 2DG can be administered for the treatment of cancer at a dose in the range of about 1 mg to about 2 g of 2-DG or 2-DGA per kg of body weight of the patient to be treated. In another embodiment, 2-DG or a 2-DGA is administered in a dose in the range of about 10 mg to about 1 g of 2-DG or a 2-DGA per kg of body weight of the patient to be treated. In certain other embodiments, 2-DG or a 2-DGA is administered in a dose of about 50 to 250 mg of a 2-DG or a 2-DGA per kg of body weight of the patient to be treated. In another embodiment, the therapeutically effective dose is about 25 mg/kg to about 150 mg/kg. For illustration, the therapeutically effective dose of 2DG or a 2DGA is administered daily or once every other day or once a week to the patient, and multiple administrations of the drug are employed. In one embodiment, 2DG or a 2DGA is administered with lonidamine or a lonidamine analog once (qday), twice (bid), three times (tid), or four times (qid) a day or once every other day (qod) or once a week (qweek), and treatment is continued for a period ranging from three days to two weeks or longer. In one embodiment, the treatment is continued for one to three months.

In another embodiment, lonidamine or a lonidamine analog is administered with an anti-angiogenic agent, including but not limited to anti-angiogenic agents selected from the group consisting of angiostatin, an agent that inhibits or otherwise antagonizes the action of VEGF, batimastat, captopril, cartilage derived inhibitor, genistein, endostatin, interleukin, lavendustin A, medroxypregesterone acetate, recombinant human platelet factor 4, Taxol, tecogalan, thalidomide, thrombospondin, TNP-470, and Avastin. Other useful angiogenesis inhibitors for purposes of the combination therapies provided by the present invention include Cox-2 inhibitors like celecoxib (Celebrex), diclofenac (Voltaren), etodolac (Lodine), fenoprofen (Nalfon), indomethacin (Indocin), ketoprofen (Orudis, Oruvail), ketoralac (Toradol), oxaprozin (Daypro), nabumetone (Relafen), sulindac (Clinoril), tolmetin (Tolectin), rofecoxib (Vioxx), ibuprofen (Advil), naproxen (Aleve, Naprosyn), aspirin, and acetaminophen (Tylenol). In addition, because pyruvic acid plays an important role in angiogenesis, pyruvate mimics and glycolytic inhibitors like halopyruvates, including bromopyruvate, can be used in combination with an anti-angiogenic compound and lonidamine or a lonidamine analog to treat cancer. In another embodiment, lonidamine or a lonidamine analog is administered with an anti-angiogenic agent and another anti-cancer agent, including but not limited to a cytotoxic agent selected from the group consisting of alkylators, Cisplatin, Carboplatin, and inhibitors of microtubule assembly, to treat cancer.

In addition to the combination of lonidamine or a lonidamine analog with the agents described above, the present invention provides a variety of synergistic combinations of lonidamine or a lonidamine analog and other anti-cancer drugs. Those of skill in the art can readily determine the anti-cancer drugs that act “synergistically” with lonidamine or a lonidamine analog as described herein. For example, the reference Vendetti, “Relevance of Transplantable Animal-Tumor Systems to the Selection of New Agents for Clinical Trial,” Pharmacological Basis of Cancer Chemotherapy, Williams and Wilkins, Baltimore, 1975, and Simpson Herren et al., 1985, “Evaluation of In Vivo Tumor Models for Predicting Clinical Activity for Anticancer Drugs,” Proc. Am. Assoc. Cancer Res. 26: 330, each of which is incorporated herein by reference, describe methods to aid in the determination of whether two drugs act synergistically. While synergy is not required for therapeutic benefit in accordance with the methods of the invention, synergy can improve therapeutic outcome. Two drugs can be said to possess therapeutic synergy if a combination dose regimen of the two drugs produces a significantly better tumor cell kill than the sum of the single agents at optimal or maximum tolerated doses. The “degree of synergy” can be defined as net log of tumor cell kill by the optimum combination regimen minus net log of tumor cell kill by the optimal dose of the most active single agent. Differences in cell kill of greater than ten-fold (one log) are considered conclusively indicative of therapeutic synergy.

When lonidamine or a lonidamine analog is used with another anti-cancer agent, the lonidamine or a lonidamine analog will, at least in some embodiments, be administered prior to the initiation of therapy with the other drug or drugs and administration will typically be continued throughout the course of treatment with the other drug or drugs. In some embodiments, the drug co-administered with lonidamine or a lonidamine analog will be delivered at a lower dose, and optionally for longer periods, than would be the case in the absence of lonidamine or a lonidamine analog administration. Such “low dose” therapies can involve, for example, administering an anti-cancer drug, including but not limited to paclitaxel, docetaxel, doxorubicin, cisplatin, or carboplatin, at a lower than approved dose and for a longer period of time together with lonidamine or a lonidamine analog administered in accordance with the methods of the present invention. These methods can be used to improve patient outcomes over currently practiced therapies by more effectively killing cancer cells or stopping cancer cell growth as well as diminishing unwanted side effects of the other therapy. In other embodiments, the other anti-cancer agent or agents will be administered at the same dose levers used when lonidamine or a lonidamine analog is not co-administered. Thus, when employed in combination with lonidamine or a lonidamine analog, the additional anti-cancer agent(s) are dosed using either the standard dosages employed for those agents when used without lonidamine or a lonidamine analog or are less than those standard dosages. The administration of lonidamine or a lonidamine analog in accordance with the methods of the invention can therefore allow the physician to treat cancer with existing (or later approved) drugs at lower doses (than currently used), thus ameliorating some or all of the toxic side effects of such drugs. The exact dosage for a given patient varies from patient to patient, depending on a number of factors including the drug combination employed, the particular disease being treated, and the condition and prior history of the patient, but can be determined using only the skill of the ordinarily skilled artisan in view of the teachings herein.

As noted above, when lonidamine is used in accordance with the methods herein in combination with another anti-cancer agent, LND will in some embodiments be first administered prior to, concurrently with, or after the initiation of therapy with the other drug or drugs and will, in any event, typically continue to be administered throughout the course of treatment with the other drug or drugs. In a preferred embodiment, LND will be administered simultaneously with the initiation of the other cancer therapy, and administration of LND will stop upon completion of the other cancer therapy. In some instances, however, the administration of LND may be temporarily halted, for days or weeks, for example, as could be the case where the side effects of LND administration were to be ameliorated.

Specific dose regimens for known and approved anticancer agents (i.e., the recommended effective dose) are known to physicians and are given, for example, in the product descriptions found in the Physician's Desk Reference 2003, (Physicians' Desk Reference, 57th Ed) Medical Economics Company, Inc., Oradell, N.J. and/or are available from the Federal Drug Administration. Illustrative dosage regimens for certain anti-cancer drugs are also provided herein. Exemplary chemotherapeutic agent(s) useful in the methods of the invention include but are not limited to: aceglatone, aclacinomycins, actinomycin F(1), aldophosphamide glycoside, altretamine, aminolevulinic acid, amsacrine, ancitabine, anthramycin, L-asparaginase, azacitidine, azaserine, 6-azauridine, benzodepa, bestrabucil, bisantrene, bleomycin, busulfan, cactinomycin, carboplatin, carboquone, carmofur, carmustine, carubicin, carzinophilin, chlorambucil, chlornaphazine, chlorozotocin, chromomycin, cisplatin, cyclophosphamide, cytarabine, dacarbazine, dactinomycin, daunomycin, daunorubicin, defofamide, demecolcine, denopterin, 2-deoxy-D-glucose, diaziquone, 6-diazo-5-oxo-1-norleucine, dideoxyuridine, doxifluridine, doxorubicin, elfornithine, elliptinium acetate, enocitabine, epirubicin, estramustine, 2-ethylhydrazide, 2,2′,2″-trichlorotriethylamine, etoglucid, etoposide, floxuridine, fludarabine, 5-fluorouracil, flutamide, fotemustine, gallium nitrate, hydroxyurea, ifosfamide, imatinib (Gleevec), improsulfan, interferon-alpha, interferon-beta, interferon-gamma, interleukin-2, lentinan, lomustine, mannomustine, mechlorethamine, mechlorethamine oxide hydrochloride, melphalan, 6-mercaptopurine, methotrexate, meturedepa, mitobronitol, mitoguazone, mitolactol, mitomycin C, mitoxantrone, mopidamol, mycophenolic acid, nimustine, nitracrine, nogalamycin, novembichin, olivomycin, paclitaxel, pentostatin, peplomycin, phenamet, phenesterine, pipobroman, piposulfan, pirarubicin, plicamycin, podophyllinic acid, porfiromycin, prednimustine, procarbazine, puromycin, pteropterin, pulmozyme, ranimustine, razoxane, sizofiran, spirogermanium, streptonigrin, streptozocin, tamoxifen, tegafur, teniposide, tenuazonic acid, thiamiprine, thioguanine, triaziquone, triethylenemelamine, triethylenephosphoramide, triethylenethiophosphoramide, trimethylolomelamine, trimetrexate, trofosfamide, tubercidin, ubenimex, uracil mustard, uredepa, urethan, vinblastine, vincristine, vindesine, zinostatin, and zorubicin,

Cancer drugs can be classified generally as alkylators, anthracyclines, antibiotics, aromatase inhibitors, bisphosphonates, cyclo-oxygenase inhibitors, estrogen receptor modulators, folate antagonists, inorganic aresenates, microtubule inhibitors, modifiers, nitrosoureas, nucleoside analogs, osteoclast inhibitors, platinum containing compounds, retinoids, topoisomerase 1 inhibitors, topoisomerase 2 inhibitors, and tyrosine kinase inhibitors. In accordance with the methods of the present invention, lonidamine or a lonidamine analog can be co-administered with any anti-cancer drug from any of these classes or can be administered prior to or after treatment with any such drug or combination of such drugs. In addition, lonidamine or a lonidamine analog can be administered in combination with a biologic therapy (e.g., treatment with interferons, interleukins, colony stimulating factors and monoclonal antibodies). Biologics used for treatment of cancer are known in the art and include, for example, trastuzumab (Herceptin), tositumomab and ¹³¹I Tositumomab (Bexxar), rituximab (Rituxan).

Alkylators useful in the practice of the present invention include but are not limited to busulfan (Myleran, Busulfex), chlorambucil (Leukeran), ifosfamide (with or without MESNA), cyclophosphamide (Cytoxan, Neosar), glufosfamide, melphalan, L-PAM (Alkeran), dacarbazine (DTIC-Dome), and temozolamide (Temodar). In accordance with the methods of the present invention lonidamine or a lonidamine analog is co-administered with an alkylator to treat cancer. In one embodiment, the cancer is chronic myelogenous leukemia, multiple myeloma, or anaplastic astrocytoma. As one example, the compound 2-bis[(2-chloroethyl)amino]tetra-hydro-2H-1,3,2-oxazaphosphorine, 2-oxide, also commonly known as cyclophosphamide, is an alkylator used in the treatment of Stages III and IV malignant lymphomas, multiple myeloma, leukemia, mycosis fungoides, neuroblastoma, ovarian adenocarcinoma, retinoblastoma, and carcinoma of the breast. Cyclophosphamide is administered for induction therapy in doses of 1500-1800 mg/m² that are administered intravenously in divided doses over a period of three to five days; for maintenance therapy, 350-550 mg/m² are administered every 7-10 days, or 110-185 mg/m² are administered intravenously twice weekly. In accordance with the methods of the invention, lonidamine or a lonidamine analog is co-administered with cyclosphosphamide administered at such doses or at lower doses and/or for a longer duration than normal for administration of cyclosphosphamide alone.

Anthracyclines useful in the practice of the present invention include but are not limited to doxorubicin (Adriamycin, Doxil, Rubex), mitoxantrone (Novantrone), idarubicin (Idamycin), valrubicin (Valstar), and epirubicin (Ellence). In accordance with the methods of the present invention lonidamine or a lonidamine analog is co-administered with an anthracycline to treat cancer. In one embodiment, the cancer is acute nonlymphocytic leukemia, Kaposi's sarcoma, prostate cancer, bladder cancer, metastatic carcinoma of the ovary, and breast cancer. As one example the compound (8S,10S)-10-[(3-Amino-2,3,6-trideoxy-.alpha.-L-lyxo-hexopyranosyl)oxy]-8-glycoloyl-7,8,9,10-tetrahydro-6,8,11-trihydroxy-1-methoxy-5,12-naphthacenedione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumor, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma. Doxorubicin is typically administered in a dose in the range of 30-75 mg/m² as a single intravenous injection administered at 21-day intervals; weekly intravenous injection at doses of 20 mg/m²; or 30 mg/m² doses on each of three successive days repeated every four weeks. In accordance with the methods of the invention, lonidamine or a lonidamine analog is co-administered starting prior to and continuing after the administration of doxorubicin at such doses (or at lower doses).

Antibiotics useful in the practice of the present invention include but are not limited to dactinomycin, actinomycin D (Cosmegen), bleomycin (Blenoxane), daunorubicin, and daunomycin (Cerubidine, DanuoXome). In accordance with the methods of the present invention lonidamine or a lonidamine analog is co-administered with an antibiotic to treat cancer. In one embodiment, the cancer is a cancer selected from the group consisting of acute lymphocytic leukemia, other leukemias, and Kaposi's sarcoma.

Aromatase inhibitors useful in the practice of the present invention include but are not limited to anastrozole (Arimidex) and letroazole (Femara). In accordance with the methods of the present invention lonidamine or a lonidamine analog is co-administered with an aromatase inhibitor to treat cancer. In one embodiment, the cancer is breast cancer.

Bisphosphonate inhibitors useful in the practice of the present invention include but are not limited to zoledronate (Zometa). In accordance with the methods of the present invention lonidamine or a lonidamine analog is co-administered with a biphosphonate inhibitor to treat cancer. In one embodiment, the cancer is a cancer selected from the group consisting of multiple myeloma, bone metastases from solid tumors, or prostate cancer.

Cyclo-oxygenase inhibitors useful in the practice of the present invention include but are not limited to celecoxib (Celebrex). In accordance with the methods of the present invention lonidamine or a lonidamine analog is co-administered with a cyclo-oxygenase inhibitor to treat cancer. In one embodiment, the cancer is colon cancer or a pre-cancerous condition known as familial adenomatous polyposis.

Estrogen receptor modulators useful in the practice of the present invention include but are not limited to tamoxifen (Nolvadex) and fulvestrant (Faslodex). In accordance with the methods of the present invention lonidamine or a lonidamine analog is co-administered with an estrogen receptor modulator to treat cancer. In one embodiment, the cancer is breast cancer or the treatment is administered to prevent the occurrence or reoccurrence of breast cancer.

Folate antagonists useful in the practice of the present invention include but are not limited to methotrexate and tremetrexate. In accordance with the methods of the present invention lonidamine or a lonidamine analog is co-administered with a folate antagonist to treat cancer. In one embodiment, the cancer is osteosarcoma. As one example, the compound N-[4-[[(2,4-diamino-6-pteridinyl)methyl methylamino]benzoyl]-L-glutamic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides. Methotrexate is administered as follows. For choriocarcinoma, intramuscular injections of doses of 15 to 30 mg are administered daily for a five-day course, such courses repeated as needed with rest period of one or more weeks interposed between courses of therapy. For leukemias, twice weekly intramuscular injections are administered in doses of 30 mg/m². For mycosis fungoides, weekly intramuscular injections of doses of 50 mg or, alternatively, of 25 mg are administered twice weekly. In accordance with the methods of the invention, lonidamine or a lonidamine analog is co-administered with methotrexate administered at such doses (or at lower doses). 5-Methyl-6-[[(3,4,5-trimethoxyphenyl)-amino]methyl]-2,4-quinazolinediamine (commonly known as trimetrexate) is another antifolate drug that can be co-administered with lonidamine or a lonidamine analog.

Inorganic arsenates useful in the practice of the present invention include but are not limited to arsenic trioxide (Trisenox). In accordance with the methods of the present invention lonidamine or a lonidamine analog is co-administered with an inorganic arsenate to treat cancer. In one embodiment, the cancer is refractory acute promyelocytic leukemia (APL).

Microtubule inhibitors (as used herein, a “microtubule inhibitor” is any agent that interferes with the assembly or disassembly of microtubules) useful in the practice of the present invention include but are not limited to vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. In accordance with the methods of the present invention lonidamine or a lonidamine analog is co-administered with a microtubule inhibitor to treat cancer. In one embodiment, the cancer is ovarian cancer, breast cancer, non-small cell lung cancer, Kaposi's sarcoma, and metastatic cancer of breast or ovary origin. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease, lymphosarcoma, reticulum-cell sarcoma, rhabdomyosarcoma, neuroblastoma, and Wilm's tumor. Vincristine is administered in weekly intravenous doses of 2 mg/m² for children and 1.4 mg/m² for adults. In accordance with the methods of the invention, lonidamine or a lonidamine analog is co-administered with vincristine administered at such doses. In one embodiment, lonidamine or a lonidamine analog is not administered prior to treatment with a microtubule inhibitor, such as a taxane, but rather, administration of lonidamine or a lonidamine analog is administered simultaneously with or within a few days to a week after initiation of treatment with a microtubule inhibitor.

In one preferred embodiment, the microtubule inhibitor used in combination with lonidamine or a lonidamine analog in the methods of the present invention is one of the taxane drugs (paclitaxel or docetaxel, for example) is co-administered with lonidamine to treat cancer. Taxol (paclitaxel) can be obtained via a semi-synthetic process from Taxus baccata. The chemical name for paclitaxel is 5^{˜},20-Epoxy-1,2^{˜},4,7^{˜},10^{˜},13^{˜}-hexahydroxytax-11-en-9-one 4,10-diacetate 2-benzoate 13-ester with (2R,3S)-N-benzoyl-3-phenylisoserine. Paclitaxel is indicated for use in combination with cisplatin for the first-line treatment of non-small cell lung cancer in patients who are not candidates for surgery and/or radiation therapy and for the treatment of breast cancer after failure of combination chemotherapy for metastatic disease or relapse within 6 months of adjuvant chemotherapy. Other approved indications for paclitaxel include: treatment of patients with metastatic carcinoma of the ovary after failure of first-line or subsequent chemotherapy; first line treatment of ovarian cancer with 3 hour infusion; adjuvant treatment of node-positive breast cancer administered sequentially to standard doxorubicin-containing combination therapy; for patients who have failed initial or subsequent chemotherapy for metastatic carcinoma of the ovary; second line therapy for AIDS related Kaposi's sarcoma; and first-line therapy for the treatment of advanced carcinoma of the ovary in combination with cisplatin. In accordance with the methods of the invention, LND is co-administered with paclitaxel and optionally other anti-cancer agents, including but not limited to 5-fluorouracil or a prodrug thereof such as Xeloda, marketed by Roche, for any of these indications.

Another microtubule inhibitor, docetaxel, is an anti-neoplastic agent belonging to the taxoid family. It can be prepared by semi-synthesis starting with a precursor extracted from the needle biomass of yew plants. The chemical name for docetaxel is (2R,3S)-N-carboxy-3-phenylisoserine, N-tert- butyl ester, 13-ester with 5b-20-epoxy-1,2a,4,7b,10b,13a-hexahydroxytax-11-en-9-one 4-acetate 2-benzoate, trihydrate. It has been approved for treating locally advanced or metastatic non-small cell lung cancer after failure of prior platinum-based chemotherapy. It has also been indicated for the treatment of locally advanced or metastatic breast cancer which has progressed during anthracycline-based treatment or relapsed during anthracycline-based adjuvant therapy. In accordance with the methods of the invention, LND is co-administered with docetaxel (or another compound in the taxoid family, such as paclitaxel) and optionally other anti-cancer agents, including but not limited to 5-fluorouracil or a prodrug thereof such as Xeloda, marketed by Roche, for any of these indications.

Other microtubule inhibitors useful in combination with lonidamine or a lonidamine analog include the vinca alkaloids. In one embodiment, vinorelbine is co-administered with lonidamine or a lonidamine analog to treat cancer. Vinorelbine (Navelbine) was approved for use as a single agent or in combination with cisplatin for the first-line treatment of ambulatory patients with non-resectable, advanced non-small cell lung cancer. It inhibits spindle formation during mitosis and cell division. In accordance with the one method of the invention, lonidamine or a lonidamine analog is co-administered with vinorelbine and optionally other anti-cancer agents, including but not limited to cisplatin, for the treatment of non-small cell lung cancer. In another embodiment, cisplatin or carboplatin and a vinca alkaloid are used in combination with lonidamine to treat multidrug-resistant tumors or to treat tumors and cancers that are refractory to treatment.

Modifiers useful in the practice of the present invention include but are not limited to Leucovorin (Wellcovorin), which is used with other drugs such as 5-fluorouracil to treat colorectal cancer. In accordance with the methods of the present invention lonidamine or a lonidamine analog is co-administered with a modifier and another anti-cancer agent to treat cancer. In one embodiment, the cancer is colon cancer. In one embodiment, the modifier is a compound that increases the ability of a cell to take up glucose, including but not limited to the compound N-hydroxyurea. In one embodiment, lonidamine or a lonidamine analog is co-administered with the metabolic inhibitor 2-deoxyglucose (2-DG) together with N-hydroxyurea. N-hydroxyurea has been reported to enhance the ability of a cell to take up 2-deoxyglucose (see the reference Smith et al., 1999, Cancer Letters 141: 85, incorporated herein by reference), and administration of N-hydroxyurea at levels reported to increase 2-DG uptake or to treat leukemia together with administration of 2-DG and lonidamine or a lonidamine analog as described herein is a therapeutic method provided by the invention. In another such embodiment, lonidamine or a lonidamine analog and 2-DG are co-administered with nitric oxide or a nitric oxide precursor, such as an organic nitrite or a spermineNONOate, to treat cancer, as the latter compounds stimulate the uptake of glucose and so stimulate the uptake of 2-DG.

Nitrosoureas useful in the practice of the present invention include but are not limited to procarbazine (Matulane), lomustine, CCNU (CeeBU), carmustine (BCNU, BiCNU, Gliadel Wafer), and estramustine (Emcyt). In accordance with the methods of the present invention, lonidamine or a lonidamine analog is co-administered with a nitrosourea to treat cancer. In one embodiment, the cancer is prostate cancer or glioblastoma, including recurrent glioblastoma multiforme.

Nucleoside analogs useful in the practice of the present invention include but are not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda). In accordance with the methods of the present invention, lonidamine or a lonidamine analog is co-administered with a nucleoside analog to treat cancer. In one embodiment, the cancer is B-cell lymphocytic leukemia (CLL), hairy cell leukemia, adenocarcinoma of the pancreas, metastatic breast cancer, non-small cell lung cancer, or metastatic colorectal carcinoma. As one example, the compound 5-fluoro-2,4(1H,3H)-pyrimidinedione, also commonly known as 5-fluorouracil, is an antimetabolite nucleoside analog effective in the palliative management of carcinoma of the colon, rectum, breast, stomach, and pancreas in patients who are considered incurable by surgical or other means. 5-Fluorouracil is administered in initial therapy in doses of 12 mg/m² given intravenously once daily for 4 successive days with the daily dose not exceeding 800 mg. If no toxicity is observed at any time during the course of the therapy, 6 mg/kg are given intravenously on the 6th, 8th, 10th, and 12th days. No therapy is given on the 5th, 7th, 9th, or 11th days. In poor risk patients or those who are not in an adequate nutritional state, a daily dose of 6 mg/kg is administered for three days, with the daily dose not exceeding 400 mg. If no toxicity is observed at any time during the treatment, 3 mg/kg may be given on the 5th, 7th, and 9th days. No therapy is given on the 4th, 6th, or 8th days. A sequence of injections on either schedule constitutes a course of therapy. In accordance with the methods of the invention, lonidamine or a lonidamine analog is co-administered with 5-FU administered at such doses or with the prodrug form Xeloda with correspondingly adjusted doses. As another example, the compound 2-amino-1,7-dihydro-6H-purine-6-thione, also commonly known as 6-thioguanine, is a nucleoside analog effective in the therapy of acute non-pymphocytic leukemias. 6-Thioguanine is orally administered in doses of about 2 mg/kg of body weight per day. The total daily dose may be given at one time. If after four weeks of dosage at this level there is no improvement, the dosage may be cautiously increased to 3 mg/kg/day. In accordance with the methods of the invention, lonidamine or a lonidamine analog is co-administered with 6-TG administered at such doses (or at lower doses).

Another nucleoside analogue preferred for use in the combination therapies of the present invention is Gemcitabine. Gemcitabine is 2′-deoxy-2′,2′-difluoro-cytidine. It is commercially available as the monohydrochloride salt, and as the beta-isomer. It is also known chemically as 1-(4-amino-2-oxo-1-H-pyrimidin-1-yl)-2-desoxy-2,2-difluororibose. Gemcitabine is disclosed in U.S. Pat. Nos. 4,808,614 and 5,464,826, incorporated herein by reference for their teaching of how to synthesize, formulate, and use gemcitabine for treating susceptible neoplasms. The commercial formulation of gemcitabine hydrochloride is indicated as first-line treatment for patients with locally advanced (non-resectable Stage II or Stage III) or metastatic (Stage IV) adenocarcinoma of the pancreas, and is commonly used in patients previously treated with 5-fluorouracil. It has also been indicated for use in combination with cisplatin for the first-line treatment of patients with inoperable, locally advanced (stage IIIA or IIIB) or metastatic (Stage IV) non-small cell lung cancer. The method of treating cancer by administration of a combination of lonidamine or a lonidamine analog and gemcitabine provided by the present invention can also be practiced with the administration of additional anti-cancer drugs, including but not limited to, mitomycin C.

Osteoclast inhibitors useful in the practice of the present invention include but are not limited to pamidronate (Aredia). In accordance with the methods of the present invention, lonidamine or a lonidamine analog is co-administered with an osteoclast inhibitor to treat cancer. In one embodiment, the cancer is osteolytic bone metastases of breast cancer, and one or more additional anti-cancer agents are also co-administered with lonidamine or a lonidamine analog.

Platinum compounds useful in the practice of the present invention include but are not limited to cisplatin (Platinol) and carboplatin (Paraplatin). In accordance with the methods of the present invention, lonidamine or a lonidamine analog is co-administered with a platinum compound to treat cancer. In one embodiment, the cancer is metastatic testicular cancer, metastatic ovarian cancer, ovarian carcinoma, and transitional cell bladder cancer. As one example, the compound cis-diamine-dichloroplatinum (II), commonly known as cisplatin, is useful in the palliative treatment of metastatic testicular and ovarian tumors, and for the treatment of transitional cell bladder cancer which is not amenable to surgery or radiotherapy. Cisplatin, when used for advanced bladder cancer, is administered in intravenous injections of doses of 50-70 mg/m² once every three to four weeks. In accordance with the methods of the present invention, lonidamine or a lonidamine analog is co-administered with cisplatin administered at these doses (or at lower doses). One or more additional anti-cancer agents can be co-administered with the platinum compound and lonidamine or a lonidamine analog. As one example, Platinol, Blenoxane, and Velbam may be co-administered with lonidamine or a lonidamine analog. As another example, Platinol and Adriamycin may be co-administered with lonidamine or a lonidamine analog.

Retinoids useful in the practice of the present invention include but are not limited to tretinoin, ATRA (Vesanoid), alitretinoin (Panretin), and bexarotene (Targretin). In accordance with the methods of the present invention, lonidamine or a lonidamine analog is co-administered with a retinoid to treat cancer. In one embodiment, the cancer is a cancer selected from the group consisting of APL, Kaposi's sarcoma, and T-cell lymphoma.

Topoisomerase 1 inhibitors useful in the practice of the present invention include but are not limited to topotecan (Hycamtin) and irinotecan (Camptostar). In accordance with the methods of the present invention, lonidamine or a lonidamine analog is co-administered with a topoisomerase 1 inhibitor to treat cancer. In one embodiment, the cancer is metastatic carcinoma of the ovary, colon, or rectum, or small cell lung cancer. In another method of the invention, topotecan is administered in combination with lonidamine to treat cancer. Topotecan (Hycamtin) is (S)-10-[(dimethylamino)methyl]-4-ethyl-4,9-dihydroxy-1H-pyrano[3′,4′:6,7]indolizino[1,2-b]quinoline-3,14-(4 H,12 H )-dione monohydrochloride and was approved for the treatment of small cell lung cancer sensitive disease after failure of first-line chemotherapy. In clinical studies submitted to support approval, sensitive disease was defined as disease responding to chemotherapy but subsequently progressing at least 60 days (in the phase 3 study) or at least 90 days (in the phase 2 studies) after chemotherapy. Another approved use is for the treatment of patients with metastatic carcinoma of the ovary after failure of initial or subsequent chemotherapy. In accordance with the methods of the invention, LND or a lonidamine analog is co-administered with topotecan and optionally other anti-cancer agents, including but not limited to doxorubicin, for any of these indications.

Topoisomerase 2 inhibitors useful in the practice of the present invention include but are not limited to etoposide, VP-16 (Vepesid), teniposide, VM-26 (Vumon), and etoposide phosphate (Etopophos). In accordance with the methods of the present invention lonidamine or a lonidamine analog is co-administered with a topoisomerase 2 inhibitor to treat cancer. In one embodiment, the cancer is a cancer selected from the group consisting of refractory testicular tumors, refractory acute lymphoblastic leukemia (ALL), and small cell lung cancer.

Tyrosine kinase inhibitors useful in the practice of the present invention include but are not limited to imatinib (Gleevec). In accordance with the methods of the present invention, lonidamine or a lonidamine analog is co-administered with a tyrosine kinase inhibitor to treat cancer. In one embodiment, the cancer is CML or a metastatic or unresectable malignant gastrointestinal stromal tumor.

Thus, the present invention provides methods of treating cancer in which lonidamine or a lonidamine analog or a pharmaceutically acceptable salt thereof and one or more additional anti-cancer agents are administered to a patient. Specific embodiments of such other anti-cancer agents include without limitation 5-methyl-6-[[(3,4,5-trimethoxyphenyl)amino]-methyl]-2,4-quinazolinediamine or a pharmaceutically acceptable salt thereof, (8S,10S)-10-(3-amino-2,3,6-trideoxy-alpha-L-lyxo-hexopyranosyl)oxy]-8-glycoloyl-7,8,9,10-tetrahydro-6,8,11-trihydroxy-1-methoxy-5,12-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]-methylamino]benzoyl]-L-glutamic acid, or a pharmaceutically acceptable salt thereof; or cis-diamminedichloroplatinum (II).

Thus, a wide variety of anticancer agents can be used in the methods of the invention. Lonidamine and lonidamine analogs can be administered to a cancer patient in combination with other agents or procedures intended to treat cancer, ameliorate symptoms of cancer, potentiate the effects of the lonidamine or lonidamine analog, or provide other therapeutic benefit. Administration of an agent “in combination with” includes parallel administration (administration of both the agents to the patient over a period of time, such as administration of lonidamine and 2-DG on alternate days for one month), co-administration (in which the agents are administered at approximately the same time, e.g., within about a few minutes to a few hours of one another), and co-formulation (in which the agents are combined or compounded into a single dosage form suitable for oral or parenteral administration). In one embodiment of the invention lonidamine or a lonidamine analog is coadministered in combination with an anti-cancer agent other than 2-DG.

The methods of the present invention are generally applicable to all cancers but have particularly significant therapeutic benefit in the treatment of solid tumors, which are characterized by extensive regions of hypoxic tissue. Particular cancers that can be treated with the methods of the present invention are discussed in the following section.

(v) Treatment of Particular Cancers

The methods and compositions of the present invention are generally applicable to all cancers but have particularly significant therapeutic benefit in the treatment of solid tumors, which are characterized by extensive regions of hypoxic issue. Such cancers include but are not limited to non-small cell lung cancer, head and neck squamous cancer, prostate cancer, ovarian cancer, colorectal cancer, and breast cancer. In one important embodiment, the methods of the invention comprise administering an anti-neoplastically effective amount of lonidamine or a lonidamine analog or a pharmaceutically acceptable salt thereof in combination with an antineoplastically effective amount of one or more additional anti-cancer compounds, and/or radiation therapy, and/or surgery.

The methods and compositions of the present invention can be used to treat the most common cancers, including but not limited to bladder cancer, breast cancer, colorectal cancer, endometrial cancer, head and neck cancer, leukemia, lung cancer, lymphoma, melanoma, non-small cell lung cancer, ovarian cancer, and prostate cancer. In one embodiment, the cancer is a cancer other than breast, cervical, prostate, and lung. In another embodiment, the cancer is a cancer selected from the group consisting of breast, cervical, prostate, and lung cancers, and lonidamine or a lonidamine analog is administered with another metabolic inhibitor or anticancer agent to treat the cancer.

The methods and compositions of the present invention can also be used to treat less common cancers, including but not limited to acute lymphocytic leukemia, adult acute myeloid leukemia, adult non-Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and small-cell lung cancer. Childhood cancers amenable to treatment by the methods and with the compositions of the present invention include but are not limited to brain stem glioma, cerebellar astrocytoma, cerebral astrocytoma, ependymoma, Ewing's sarcoma and family of tumors, germ cell tumor—extracranial, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, and Wilms's tumor and other childhood kidney tumors.

The methods and compositions of the present invention can also be used to treat cancers that have originated in or metastasized to the bone, brain, breast, digestive and gastrointestinal systems, endocrine system, eye, genitourinary tract, germ cells, gynecological system, head and neck, hematologic system, blood, lung, respiratory system, thorax, musculoskeletal system, and skin.

In one preferred embodiment of the invention, lonidamine or a lonidamine analog is used in combination with another anti-cancer agent to treat non-small-cell lung cancer. Current treatment regimens for non-small-cell lung cancer include administration of Gemcitabine, Vinorelbine, Paclitaxel, Docetaxel, cisplatin, carboplatin, or Irinotecan as single agents; and administration of Etoposide and cisplatin, Vindesine and cisplatin, Paclitaxel and carboplatin, Gemcitabine and carboplatin, Docetaxel and cisplatin, Vinorelbine and cisplatin, or Irinotecan and cisplatin in combination therapies. See Bunn, 15 Sep. 2002, J. Clin. Onc. 20(18s): 23-33, incorporated herein by reference. In accordance with the methods of the present invention, lonidamine can be co-administered in such therapeutic regimens to improve patient outcomes. In another preferred embodiment of the invention, the combination of lonidamine with 2-deoxy-D-glucose is administered with the anti-cancer agents used therapeutic regimens for the treatment of non-small cell lung cancer. In another embodiment of the invention, a combination of (i) lonidamine, and (ii) either cisplatin or carboplatin, together with (iii) either taxol, taxotere, gemcitabine, and vinrelbine are administered to a patient with non-small-cell lung cancer to treat the disease. Each drug is administered at a dose known in the art for the treatment of cancer.

In one embodiment, the cancer to be treated is lung cancer or non-small-cell lung (NSCL) cancer, and lonidamine or a lonidamine analog is administered in combination with a taxane and a platinum-containing anticancer agent, including without limitation Taxol and cisplatin, Taxol and carboplatin, and Taxotere and cisplatin. In another embodiment, the cancer to be treated is lung cancer or non-small-cell lung cancer, and lonidamine or a lonidamine analog is administered in combination with a nucleoside analog and a platinum-containing anticancer agents, including without limitation Gemcitabine and cisplatin, and Gemcitabine and carboplatin. In another embodiment, the cancer to be treated is lung cancer or non-small-cell lung cancer, and an EGF receptor antagonist, including without limitation Tarceva and Irbitux (C225), is used in combination with one of the aforementioned combination therapies. In another embodiment, the cancer is relapsed NSCL, and the treatment is administration of Taxotere and lonidamine or a lonidamine analog.

In another embodiment of the invention, a combination of (i) lonidamine or a lonidamine analog, and (ii) either (a) a taxane such as Taxol or Taxotere, or (b) cytoxan, together with (iii) either (c) herceptin if Taxol is administered or (d) 5-fluorouracil and either adriamycin or methotrexate if cytoxan is administered, are administered to a patient with breast cancer to treat the disease. Each drug is administered at a dose known in the art for the treatment of cancer. In one embodiment, lonidamine or a lonidamine analog is co-administered with an angiogenesis inhibitor to treat breast cancer. In one embodiment the angiogenesis inhibitor is avastin.

In another embodiment of the invention, a combination of (i) lonidamine or a lonidamine analog, and (ii) either prednisone or taxotere, and optionally with mitoxantrone if prednisone is administered, are administered to a patient with prostate cancer to treat the disease. Each drug is administered at a dose known in the art for the treatment of cancer. In one aspect, the present invention provides a new pharmaceutical formulation comprising lonidamine and prednisone and one or more pharmaceutically acceptable carriers or excipients suitable for oral administration. In another embodiment, lonidamine or a lonidamine analog is administered alone or in combination with another anti-cancer agent, surgery, and/or radiation to treat relapsing, hormone-dependent prostate cancer, wherein the relapsing patient has rising PSA levels.

In another embodiment of the invention, a combination of (i) lonidamine or a lonidamine analog, and (ii) either (a) captosar, or (b) 5-fluorouracil and levamisole are administered to a patient with colorectal cancer to treat the disease. In another embodiment, colorectal cancer is treated by administering lonidamine or a lonidamine analog and Avastin. In another embodiment, colorectal cancer is treated by administering lonidamine or a lonidamine analog and Avastin and oxaliplatin. In another embodiment, colorectal cancer is treated by administering lonidamine or a lonidamine analog and Avastin and 5-FU and levamisole. In another embodiment, colorectal cancer is treated by administering lonidamine or a lonidamine analog and Avastin and CPT-11, optionally including in addition 5-FU and levamisole. In another embodiment, colorectal cancer is treated by administering lonidamine or a lonidamine analog and an EGF receptor inhibitor, including but not limited to Tarceva and Irbitux. Each drug is administered at a dose known in the art for the treatment of cancer.

In another embodiment of the invention, a combination of (i) lonidamine or a lonidamine analog, and (ii) either cisplatin or carboplatin or another platinum containing anti-cancer agent, together with (iii) either taxol or taxotere or another taxane are administered to a patient with ovarian cancer to treat the disease. Each drug is administered at a dose known in the art for the treatment of cancer.

In another embodiment of the invention, a combination of (i) lonidamine or a lonidamine analog, and (ii) either (a) cisplatin or carboplatin or another platinum containing anti-cancer agent, or (b) cytoxan, vincristine, and prednisone, and optionally together with adriamycin are administered to a patient with non-Hodgkin's lymphoma to treat the disease. Each drug is administered at a dose known in the art for the treatment of cancer.

In another embodiment of the invention, a combination of lonidamine or a lonidamine analog, and either cisplatin or carboplatin or another platinum containing anti-cancer agent are administered to a patient with head and neck cancer to treat the disease. Each drug is administered at a dose known in the art for the treatment of cancer. In another embodiment, head and neck cancer is treated in accordance with the present methods by administering lonidamine and a lonidamine analog and hyperfractionated radiation (division of the total dose of radiation into smaller doses that are given more than once a day).

Moreover, in a related aspect, the present invention provides a method of treating solid tumors generally, not just head and neck cancers, by administration of lonidamine or a lonidamine analog to a patient as described herein while such patient is receiving hyperfractionated radiation therapy for treatment of such cancer.

In another embodiment of the invention, a combination of lonidamine or a lonidamine analog and Avastin are administered to a patient with renal cell carcinoma or other renal cancer to treat the disease. In one embodiment, the subject is also administered II-2. Each drug is administered at a dose known in the art for the treatment of cancer.

In another embodiment of the invention, a combination of lonidamine or a lonidamine analog and Avastin are administered to a patient with pancreatic cancer o treat the disease. In another embodiment of the invention, a combination of lonidamine or a lonidamine analog and glufosfamide or ifosfamide/Mesna are administered to a patient with pancreatic cancer to treat the disease. In another embodiment of the invention, a combination of lonidamine or a lonidamine analog and Gemcitabine are administered to a patient with pancreatic cancer to treat the disease. Each drug is administered at a dose known in the art for the treatment of cancer.

In addition to the methods for treating cancer, the present invention also provides methods for treating diseases or conditions characterized by cellular hyperproliferation, including but not limited to diseases of inflammation and auto-immune diseases, including but not limited to arthritis and psoriasis.

(vi) Formulations

The compounds used in the methods of the present invention are formulated in compositions suitable for therapeutic administration. In one embodiment, the methods of the invention are practiced with lonidamine in the unit dosage form marketed as Doridamina (by ACRAF) in Italy. New dosage forms of lonidamine are also provided. For example, the present invention provides a unit dosage pharmaceutical formulation of lonidamine that is suitable for oral administration (including tablets, capsules, caplets, and pills) and contains, in various embodiments, an amount of lonidamine in a range bounded by a lower limit of (in mg) 1, 5, 10, and 50 and an upper limit of 10, 20, 40, 50, 70 and 100 (where the higher limit is in mg and greater than the lower limit) and is especially convenient for certain low dose schedules. In an other embodiment, the unit dosage form contains an amount of drug in a range bounded by a lower limit of (in mg) 200, 300, 500 or 1000 and an upper limit of 500, 1000, 3000 or 5000 (where the higher limit is greater than the lower limit) and is especially convenient for certain high dose schedules. In yet other embodiments, the formulation contains between 100 and 200 mg of compound (e.g., 150 mg), between 200 and 5000 mg, between 200 and 1000 mg, or between 500 and 1000 mg of the compound. Lonidamine analogs can be similarly formulated.

In addition to lonidamine and/or lonidamine analogs, solid unit dosage forms of the invention generally include a pharmaceutically acceptable carrier. As used herein, “pharmaceutically acceptable carrier” refers to a solid or liquid filler, diluent, or encapsulating substance, including for example excipients, fillers, binders, and other components commonly used in pharmaceutical preparations, including, but not limited to, those described below. Methods for formulation of drugs generally are well known in the art, and the descriptions herein are illustrative and not limiting. See, e.g., Ansel et al., 1999, Pharmaceutical Dosage Forms and Drug Delivery Systems 7th ed. Lippincott Williams & Wilkins, Philadelphia: pp. 1-562; Marshall, 1979. “Solid Oral Dosage Forms,” MODERN PHARMACEUTICS, Vol. 7, (Banker and Rhodes, editors), pp. 359-427.

Hydrophilic binders suitable for use in the formulations of the invention include copolyvidone (cross-linked polyvinylpyrrolidone), polyvinylpyrrolidone, polyethylene glycol, sucrose, dextrose, corn syrup, polysaccharides (including acacia, guar, and alginates), gelatin, and cellulose derivatives (including HPMC, HPC, and sodium carboxymethylcellulose).

Water-soluble diluents suitable for use in the formulations of the invention include sugars (lactose, sucrose, and dextrose), polysaccharides (dextrates and maltodextrin), polyols (mannitol, xylitol, and sorbitol), and cyclodextrins. Non-water-soluble diluents suitable for use in the formulations of the invention include calcium phosphate, calcium sulfate, starches, modified starches, and microcrystalline cellulose.

Surfactants suitable for use in the formulations of the invention include ionic and non-ionic surfactants or wetting agents such as ethoxylated castor oil, polyglycolyzed glycerides, acetylated monoglycerides, sorbitan fatty acid esters, poloxamers, polyoxyethylene sorbitan fatty acid esters, polyoxyethylene derivfatives, nonoglycerides or ethoxylated derivatives thereof, sodium lauryl sulfate, lecithins, alcohols, and phospholipids.

Disintegrants suitable for use in the formulations of the invention include starches, clays, celluloses, alginates, gums, cross-linked polymers (PVP, sodium carboxymethyl-cellulose), sodium starch glycolate, low-substituted hydroxypropyl cellulose, and soy polysaccharides. Preferred disintegrants include a modified cellulose gum such as cross-linked sodium carboxymethylcellulose.

Lubricants and glidants suitable for use in the formulations of the invention include talc, magnesium stearate, calcium stearate, stearic acid, colloidal silicon dioxide, magnesium carbonate, magnesium oxide, calcium silicate, microcrystalline cellulose, starches, mineral oil, waxes, glyceryl behenate, polyethylene glycol, sodium benzoate, sodium acetate, sodium chloride, sodium lauryl sulfate, sodium stearyl fumarate, and hydrogenated vegetable oils. Preferred lubricants include magnesium stearate and talc and combinations thereof.

The preferred range of total mass for the tablet or capsule may be from about 40 mg to 2 g, from about 100 mg to 1000 mg, and from about 300 mg to 750 mg.

In addition, the present invention provides unit dosage forms that are sustained release formulations of lonidamine or a lonidamine analog to allow once a day (or less) oral dosing, a frequency sometimes preferred by patients over multiple day dosing. Such sustained release formulations (including tablets, capsules, caplets and pills) of the invention usually contain between 1 mg and 10 g of the active compound, with various alternative embodiments including those described above for conventional oral unit doses, such as an amount of drug in a range bounded by a lower limit of (in mg) 10, 50, and 150 and an upper limit of 100, 200, 400, 500, 700 and 1000 (where the higher limit is greater than the lower limit). In another embodiment, the unit dosage form contains an amount of drug in a range bounded by a lower limit of (in mg) 200, 300, 500, 750 or 1000 and an upper limit of 500, 1000, 2000, 3000 or 5000 (where the higher limit is greater than the lower limit).

In one embodiment, lonidamine or a lonidamine analog in the sustained release formulations (also called “modified” or “controlled” release forms) is released over a period of time greater than 6 hours, e.g., greater than 12 hours, after administration. In one embodiment, the sustained release formulation allows once a day dosing to achieve a pharmacodynamic profile therapeutically equivalent to dosing 150 mg of lonidamine three times a day.

Examples of sustained-release formulations for other drugs that can be modified in accordance with the teachings herein to be useful in the present invention are well known in the art, and are, for example, described in U.S. Pat. Nos. 5,968,651; 5,266,331; 4,970,075; 5,549,912; 5,478,577; 5,472,712; 5,356,467; 5,286,493; 6,294,195; 6,143,353; 6,143,322; 6,129,933; 6,103,261; 6,077,533; 5,958,459; and 5,672,360. Sustained-release formulations are also discussed in the scientific literature, e.g., in ORAL SUSTAINED RELEASE FORMULATIONS: DESIGN AND EVALUATION, edited by A. Yacobi and E. Halperin-Walega, Pergamon Press, 1988, which describes a variety of types of sustained-release dosage forms and drug release mechanisms, for example single unit (e.g., matrix tablets, coated tablets, capsules), multiple unit (e.g., granules, beads, micro-capsules), inert, insoluble matrix, hydrophilic gel matrix (e.g., bioadhesive, erodible, non-erodible), and ion-exchange resin sustained-release dosage forms.

In one embodiment, the present invention provides a method of treating cancer, by administering once daily to a patient in need of such treatment a sustained release tablet dosage form comprising a daily therapeutic dose of lonidamine from about 1 mg to 2 g in a hydrophilic matrix. The matrix can be, for example and without limitation, selected from the group consisting of hydroxypropylmethyl cellulose (by weight percent of about 20-40%), lactose (5-15%), microcrystalline cellulose (4-6%), and silicon dioxide (1-5%) having an average particle size ranging from 1-10 microns, often ranging from 2-5 microns, and most often ranging from about 2-3 microns.

Illustrative preferred sustained release formulations of the invention include formulations A and B in the table below.

| Formulation | | |
|---|---|---|
| (weight percentage) | A | B |
| Lonidamine (milled) | 53.8 | 53.8 |
| HPMC (Methocel K15M, CR) | 8 | 30 |
| Methyl cellulose (Methocel, K100L, CR) | 18 | 0 |
| Anydrous lactose | 12.2 | 8.2 |
| Microcrystalline cellulose (Avicel PH101) | 5 | 5 |
| Silicon dioxide (1-10 micron; Syloid 244) | 3 | 3 |
| Total Table Weight (in grams) | 1 | 1 |

The sustained release formulations of the invention may be in the form of a compressed tablet containing an intimate mixture of lonidamine and a partially neutralized pH-dependent binder that controls the rate of drug dissolution in aqueous media across the range of pH in the stomach (typically ˜2) and intestine (typically ˜5.5).

Many materials known in the pharmaceutical art as “enteric” binders and coating agents have the desired pH dissolution properties suitable for use in the sustained formulations of the invention. These include phthalic acid derivatives such as the phthalic acid derivatives of vinyl polymers and copyolymers, hydroxyalkylcellulose, alkylcelluloses, cellulose acetates, hydroxyalkylcellulose acetates, cellulose ethers, alkylcellulose acetates, and esters thereof, and polymers and copoloymers of lower alkyl acrylic acids and lower alkyl acrylates, and the partial esters thereof.

Preferred pH-dependent binder materials are methacrylic acid copolymers. Such a copolymer is commercially available from Rohm Pharma as Eudragit™ L-100-55 as a powder or L30D-55 as a 30% dispersion in water. Other pH-dependent binder materials which may be used alone or in combination include hydroxypropyl cellulose phthalate, hydroxypropyl methylcellulose phthalate, cellulose acetate phthalate, polyvinylacetate phthalate, polyvinylpyrrolidone phthalate, and the like. One or more pH-dependent binders are present in the sustained release oral dosage forms of the invention in an amount ranging from about 1 to 20 weight percent, or from 5 to 12 weight percent, or about 10%.

The pH-independent binders or viscosity enhancing agents contained in the sustained release formulations of the invention include substances such as hydroxypropyl methylcellulose, hydroxypropyl cellulose, methylcellulose, polyvinylpyrrolidone, neutral poly(meth)acrylate esters, and the like. The pH-independent binders are in an amount ranging from 1 to 10 weight percent or from 1 to 3 weight percent, or about 2%.

The sustained release formulations of the invention also contain in some embodiments one or more pharmaceutical excipients intimately mixed with the ranolazine (Lonidamine) and the pH-dependent binder, such as pH-independent binders or film-forming agent, starch, gelatin, sugars, carboxymethylcellulose, and the like, as well as other useful pharmaceutical diluents such as lactose, mannitol, dry starch, microcrystalline cellulose, and the like, and surface active agents such as polyoxyethylene sorbitan esters, sorbitan esters, and the like; and coloring agents and flavoring agents. Lubricants such as talc and magnesium stearate and tableting aids are also present.

The sustained release formulations of the invention include any of the commercially available polymers suitable for use in such formulations, including but not limited to cellulose, ethyl cellulose, methyl cellulose, carboxymethyl cellulose, hydroxypropylmethyl cellulose, hydroxypropylmethyl cellulose phthalate, hydroxypropyl cellulose, microcrystalline cellulose, sodium carboxymethyl cellulose, cellulose acetate phthalate, polyvinyl acetate phthalate, polyvinylpyrrolidone, polyethylene oxide, polyethylene glycol, zein, alginate, hypromellose phthalate, methacrylic acid copolymer, Crospovidone, silica aerogel, pregelatinized starch, corn starch, croscarmellose sodium, sodium starch glycolate, candelilla wax, paraffin wax, carnauba wax, montan glycol wax, white wax, Eudragit (polymethacrylic acid esters), Aquacoat (ethyl cellulose, cellulose acetate phthalate), Carbopol (acrylic acid polyalkeny polyether copolymer), and Macrogol (polyethylene glycol).

The sustained release formulations of the invention include formulations that are diffusion controlled, such as those that employ: (a) a reservoir system in which the drug is encapsulated in a polymeric membrane, and water diffuses through the membrane to dissolve the drug, which then diffuses out of device; (b) a monolithic (matrix) system in which the drug is suspended in a polymeric matrix and diffuses out through long pathways; (c) microencapsulation and coated granule systems in which particles of drug (or particles of drug and polymer) as small as 1 micron are coated in a polymeric membrane, including embodiments in which particles coated with polymers with different release characteristics are delivered together in a capsule; (d) solvent-activated systems, including (i) osmotically controlled devices (e.g. OROS) in which an osmotic agent and the drug are encapsulated in a semi-permeable membrane, water is pulled into device due to osmotic gradient, and increased pressure drives drug out of device through a laser drilled hole; (ii) a hydrogel swelling system in which drug is dispersed in a polymer and/or a polymer is coated onto a particle of drug, and the polymer swells on contact with water (swelling is in some embodiments pH or enyzmatically controlled), allowing diffusion of drug out of the device; (iii) a microporous membrane system in which drug is encapsulated in a membrane that has a component that dissolves on contact with water (in some embodiments, dissolution is pH or enyzmatically controlled), leaving pores in the membrane through which the drug diffuses; and (iv) a wax matrix system in which the drug and an additional soluble component are dispersed in wax, such that, when water dissolves the component, diffusion of drug from the system is allowed; and (e) polymeric degradation systems, including (i) bulk degradation, in which drug is dispersed in polymeric matrix, and degradation occurs throughout the polymeric structure in a random fashion, allowing drug release; and (ii) surface erosion, in which drug-is dispersed in polymeric matrix and delivered as the surface of the polymer erodes.

In one aspect, the invention provides a method for treating cancer by administering a unit dose oral pharmaceutical composition that is a sustained-release formulation containing an effective amount of lonidamine or a lonidamin analog, such as described above, once per day.

(vii) Theranostics

“Theranostics”, as used herein, refers to a combined diagnostic/therapeutic procedure, in which a patient is tested to determine suitability for a particular therapy prior to the administration of the therapy. The present invention provides a number of such methods to assess whether the cancer is particularly susceptible to lonidmine therapy. In one embodiment, the diagnostic step involves conducting an assay on a all or a portion of a tumor (i.e., a needle biopsy of a solid tumor) to determine if the tumor contains substantial hypoxic regions. While an oxygen sensor can be used for such an assay, other embodiments of this aspect of the invention involve assays in which the level or activity of HIF-1a is measured, because high HIF-1a levels are indicative of substantial hypoxic regions in the tumor. Other surrogate assays for this purpose include glucose transporter levels and activity (higher levels correlate with hypoxia); VegF levels or activity (higher levels correlate with hypoxia); and, as described elsewhere herein, ATP levels or the levels of other energy-providing molecules, such as NADH/NADPH (lower levels correlate with hypoxia).

Thus, the present invention provides methods for treating cancer that involve a preliminary assessment of the cancer patient to determine the degree of susceptibility of the patient's cancer to lonidamine (or lonidamine analog) mediated drug therapy. In one aspect, this assessment evaluates the hypoxic state of the tumor, because in general the more hypoxic the tumor the more susceptible the tumor to treatment with lonidamine therapy, or the energy state of the tumor, because the lower the ATP concentration in a cancer cell, the more susceptible that cell is to treatment with lonidamine or a lonidamine analog. Thus, in one embodiment, the patient's tumor is probed with an oxygen sensor to determine the hypoxic state of the tumor. In one embodiment, HIF-1alpha expression in the cancer cells in the patient is examined, as increased HIF-1alpha expression correlates with increased hypoxia. In one embodiment, the cancer cells in the patient are evaluated for the level of glucose utilization or the level of glucose transporters, as increased glucose utilization and increased glucose transport indicate increased susceptibility to treatment with lonidamine or a lonidamine analog. In one embodiment, the cancer cells of the patient are evaluated for ATP concentration or production, as low ATP levels indicate increased susceptibility to lonidamine or a lonidamine analog mediated therapy. In one embodiment, VEGF expression is measured or otherwise determined in the patient's cancer cells, as increased VEGF expression indicates increased susceptibility to lonidamine or a lonidamine analog mediated therapy.

In one embodiment, the patient's cancer cells are tested for the presence of cancer-related or cancer-causing mutations, including but not limited to p53 mutations, mutations in the VHL (Von Hippel Lindau) factor gene, mutations associated with drug resistance, as such mutations often arise in the hypoxic areas of tumors and so are indicative of a tumor highly susceptible to lonidamine mediated therapy.

Thus, the present invention provides useful compounds, compositions, and methods for treating cancer using lonidamine and lonidamine analogs alone and in combination with other anticancer agents and therapies, such as surgery and radiation.

### EXAMPLE 1

### Lonidamine Reduces Expression of HIF-1 Alpha in Prostate Cells

This example shows the effects of lonidamine treatment on HIF-1alpha expression in two cell lines derived from metastatic lesions of human prostate cancers. LNCaP is a citrate-producing cell (ATTC No. CRL-1740) while PC3 is citrate oxidizing cell (ATTC No. CRL-1435). See Franklin et al.; 1995, “Regulation of citrate metabolism by androgen in the LNCaP human prostate carcinoma cell line.” Endocrine 3:603-607. Cells may be obtained from the American Type Culture Collection (ATCC), P.O.Box 1549, Manassas, Va. 20108 USA.

The data demonstrate that lonidamine is an inhibitor of hypoxia-induced accumulation of HIF-1alpha in these cell lines under the conditions tested. In addition, citrate-producing cells (LNCaP) displayed greater sensitivity to lonidamine treatment compared to citrate-oxidizing cells (PC3). While the results of these experiments do not definitively establish the mechanism or specificity of inhibition of HIF-1alpha by lonidamine, the results do demonstrate that lonidamine can inhibit HIF-1alpha in tumor cell lines and so indicate that the combination therapies of the invention should be particularly effective in killing cells in the hypoxic regions of tumors. While lonidamine's effect on HIF-1alpha levels may be due entirely or in part to a general inhibition of protein synthesis, described as an activity of lonidamine by Floridi et al., 1985, “Effect of lonidamine on protein synthesis in neoplastic cells” Exp. Mol. Path. 42: 293-305, its effect could also be due entirely or in part to lonidamine's effect on oxygen utilization by mitochondria. Hagen et al., 12 Dec. 2003, “Redistribution of Intracellular Oxygen in Hypoxia by Nitric Oxide: Effect on HIF1” Science 302:1975-78, reported that HIF-1alpha is constitutively synthesized but degraded in the presence of oxygen. It is possible that, under hypoxic conditions, inhibition of mitochondrial respiration by lonidamine reduces oxygen consumption by mitochondria. This in turn could lead to enhanced activity of the oxygen-dependent enzyme, prolyl hydrolase, which plays a role in the HIF-1alpha degradation pathway.

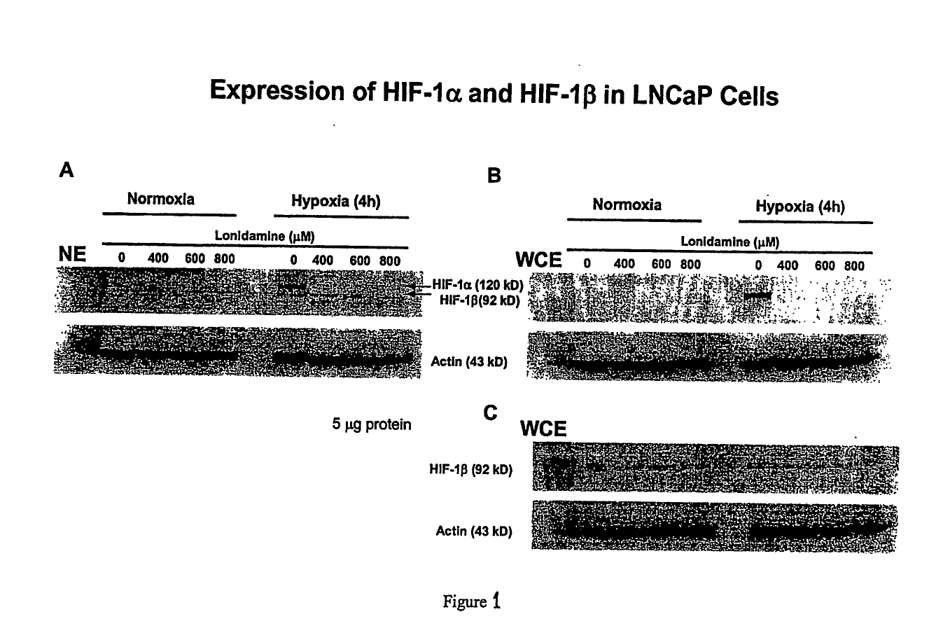

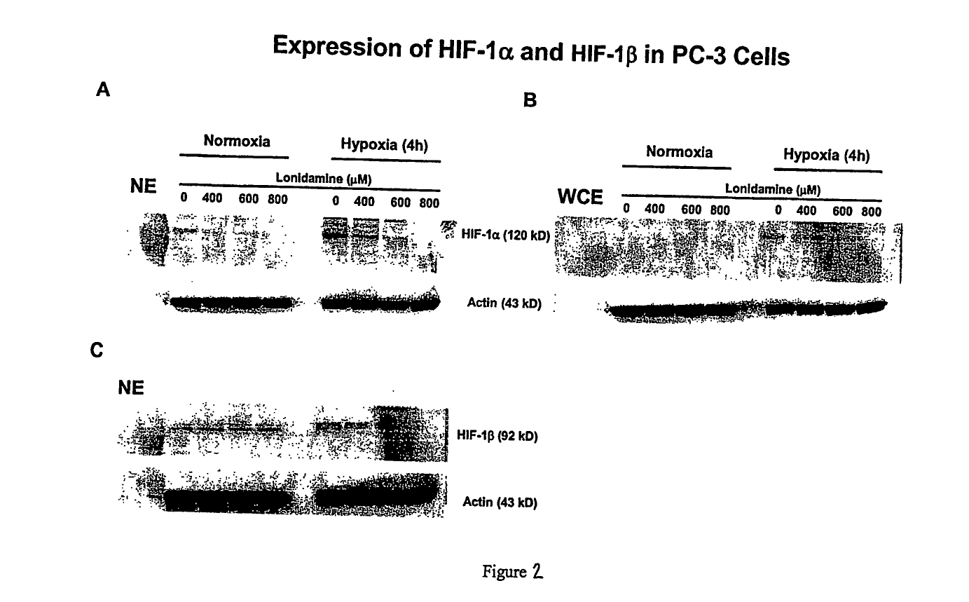

As shown in FIGS. 1 and 2, lonidamine treatment reduced the level of HIF-1alpha protein detected in nuclear (NE) and whole-cell extract (WCE) preparations. The inhibition was dose-dependent, and observed under normoxic (PC3 cells only) and hypoxic conditions (LNCaP cells and PC3 cells). The lonidamine effect was specific to the HIF-1alpha subunit under the conditions tested and, except at 800 microM concentration, had no detectable inhibition under the conditions tested on the protein levels of actin, caspase 3, NF-kappabeta, or Ikappabetaalpha.

In the event that lonidamine's inhibitory effect on HIF-1alpha occurs in tumor cells in vivo as a result of administering lonidamine (or a lonidamine analog) as described herein, then the combination therapies disclosed herein should be even more effective in treating particularly difficult-to-treat cancers, including but not limited to colorectal cancer, renal cell carcinoma, NSCL cancer, and pancreatic cancer. HIF-1alpha controls the synthesis and secretion of vascular endothelial growth factor, and inhibiting its activity should make the tumor even more hypoxic, rendering it even more sensitive to metabolic inhibition by lonidamine. As described herein, lonidamine is useful in combination with VEGF inhibitors in the treatment of tumors where inhibitors of VEGF have proven useful. Should the HIF-1alpha inhibition observed in the tests reported here also occur in cancer cells in vivo upon the administration of lonidamine, then lonidamine itself can serve as the VEGF inhibitor in such therapies. Thus, for example, lonidamine is useful in the treatment of colorectal cancer in combination with, for example, 5-FU/levamosol for advanced colorectal cancer, as well as in combination with those same drugs and a VEGF inhibitor such as Avastin. Should lonidamine's anti-HIF-1-alpha activity result in the same degree of VEGF inhibition as Avastin, then the former method of the invention can be equally efficacious to the latter in treating tumors. Likewise, lonidamine is useful for the treatment of advanced colorectal cancer in combination with CPT-11 or oxaliplatin, whether alone or in combination with a VEGF inhibitor, but again, the former method could be equally efficacious if lonidamine can specifically inhibit HIF-1alpha such that VEGF is inhibited to the same extent as if Avastin had been administered. Likewise, the methods of the invention for treating pancreatic cancer by administering lonidamine in combination with gemcitabine, and with or without a VEGF inhibitor, and for treating advanced breast cancer in combination with taxanes with or without other agents such as cisplatin and doxorubicin, and with or without a VEGF inhibitor, may be similarly efficacious. Those of skill in the art will appreciate, however, that if lonidamine is not a specific and/or efficacious inhibitor of HIF-1 alpha activity, lonidamine's therapeutic benefit in reducing energy production in cancerous cells nonetheless make lonidamine a highly useful agent in combination therapies as described treatments for all solid tumors.

Methods: Cells were plated at a density of 5×10⁵ cells into a dish, and then maintained in 37° C. incubator (5% CO₂) for 2 days. Prior to the assay, cells were rinsed twice with pre-warmed (37° C.) RPMI-1640 Medium (ATCC No. 30-2001; 10 mM HEPES; 1 mM sodium pyruvate; 2 mM L-glutamine; 4500 mg glucose/L; 1500 mg sodium bicarbonate/L). Cells were incubated with 2 mL culture medium in the absence or presence of lonidamine at different concentrations for 4 hours at 37° C. either under normoxia or hypoxia (oxygen level<0.1%). At the end of the incubation, the dish was placed on ice, and the cells were washed rapidly twice with cold PBS buffer (4° C.). For nuclear extracts, cells were lysed with buffer A (10 mM Tris, pH7.5; 1.5 mM MgCl₂; 10 mM KCl and protease inhibitors and buffer C (0.5 M NaCl; 20 mM Tris pH7.5; 1.5 mM MgCl₂; 20% glycerol and protease inhibitors), sequentially. The protease inhibitors used in the experiments were a cocktail of five protease inhibitors (500 mM AEBSF-HCl, 1 mg/ml Aprotinin, 1 mM E-64, 500 mM EDTA and 1 mM Leupeptin; Calbiochem NO 539131). For whole cell lysate, cells were lysed with 150 mM NaCl; 10 mM Tris ph7.5; 10 mM EDTA; 1% Triton X-100; 0.5% Deoxycholate, and protease inhibitors. The protein concentration was measured using a Bio-Rad protein assay. Equal amounts of protein were loaded on a SDS-PAGE gel. After transferring of the sample to PVDF membrane, the membrane was blocked with TBST containing 5% non-fat milk overnight at 4° C. Subsequently, the membrane was incubated with primary antibodies (HIF-1alpha, HIF-1beta, and actin) and alkaline phosphatase-conjugated secondary antibody, for two hours each incubation. To detect the expression of caspase 3, NF-κB, P65 and IκBalpha, the membrane was blocked with TBST containing 5% non-fat milk for 1 h at room temperature, and the proteins were detected by incubation with the corresponding antibodies overnight at 4° C. and with the alkaline phosphatase-conjugated secondary antibody for 1 h. The specific protein was detected using a colorimetric substrate, and the intensity of each protein was quantified using an NIH image system.

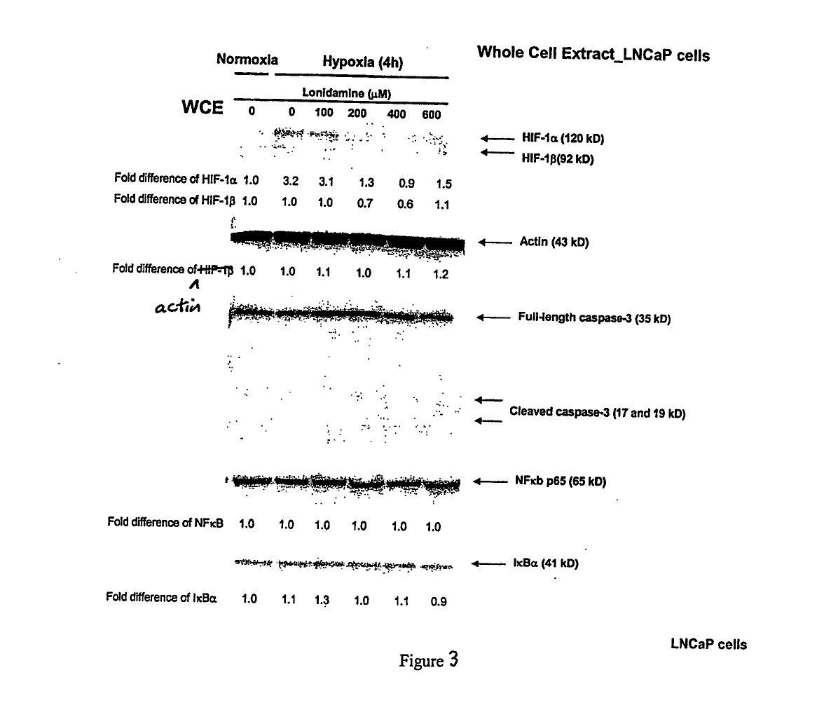

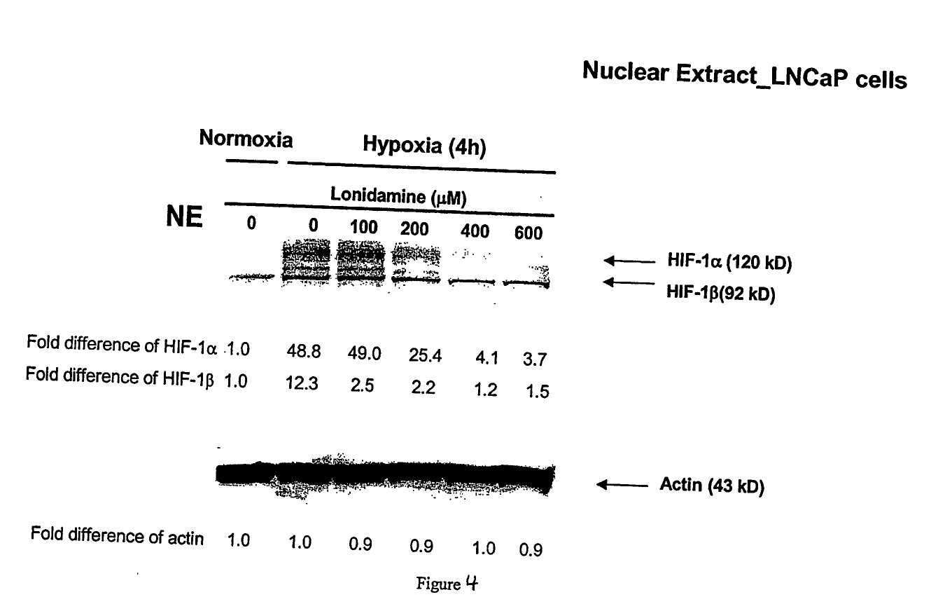

In separate experiments carried out generally as above, the effect of 0-600 microM lonidamine on expression of HIF-1alphaand other proteins was determined in LNCaP whole cell extracts (FIG. 3) or nuclear extracts (FIG. 4) from cells cultured under hypoxic conditions.

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description and examples are for purposes of illustration and not limitation of the following claims.

What is claimed:

1. A method for treating cancer, said method comprising administering to a mammal a therapeutically effective amount of lonidamine in combination with a therapeutically effective amount of one or more additional chemotherapeutic agents.

2. The method of claim 1, wherein said cancer is selected from the group consisting of leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas.

3. The method of claim 1, wherein the said chemotherapeutic agent is selected from the group consisting of busulfan, improsulfan, piposulfan, benzodepa, carboquone, 2-deoxy-D-glucose, meturedepa, uredepa, altretamine, imatinib, triethylenemelamine, triethylenephosphoramide, triethylenethiophosphoramide, trimethylolomelamine, chlorambucil, chlornaphazine, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elfornithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, teniposide, tenuazonic acid, triaziquone, 2,2′,2″-trichlorotriethylamine, urethan, vinblastine and vincristine.

4. The method of claim 3, wherein said chemotherapeutic agent is selected from the group consisting of 2-deoxy-D-glucose, paclitaxel, docetaxel, gemcitabine, and vinorelbine.

5. The method of claim 4, wherein said chemotherapeutic agent is gemcitabine.

6. The method of claim 4, wherein said chemotherapeutic agent is a taxane.

7. The method of claim 4, wherein said chemotherapeutic agent is 2-deoxy-D-glucose.

8. The method of claim 2, wherein said cancer is non-small-cell lung cancer, and lonidamine is co-administered with either cisplatin or carboplatin together with an anti-cancer agent selected from the group consisting of taxol, taxotere, gemcitabine, and vinorelbine.

9. The method of claim 2, wherein said cancer is breast cancer, and lonidamine is co-administered with either (a) taxol or taxotere and herceptin, or (b) cytoxan and 5-fluorouracil and either adriamycin or methotrexate.

10. The method of claim 2, wherein said cancer is prostate cancer, and ionidamine is co-administered with either prednisone or taxotere, and optionally with mitoxantrone if prednisone is administered.

11. The method of claim 2, wherein said cancer is colorectal cancer, and lonidamine is co-administered with either captosar or 5-fluorouracil and levamisole.

12. The method of claim 2, wherein said cancer is ovarian cancer, and lonidamine is co-administered with either cisplatin or carboplatin, together with either taxol or taxotere.

13. The method of claim 2, wherein said cancer is ovarian cancer, and lonidamine is co-administered with either (a) cisplatin or carboplatin, or (b) cytoxan, vincristine, and prednisone, and optionally together with adriamycin.

14. The method of claim 2, wherein said chemotherapeutic agent is both 2-deoxy-2-glucose and one or more agents selected from the group consisting of cisplatin, carboplatin, taxol, taxotere, cytoxan, vincristine, adriamycin, captosar, 5-fluorouracil, levamisole, prednisone, mitoxantrone, herceptin, and vinorelbine.

15. A method of treating cancer, said method comprising administering a therapeutically effective dose of lonidamine or a lonidamine analog in combination with administering hyperfractionated radiation therapy.

16. The method of claim 16, wherein said cancer is head and neck cancer.

17. A method of treating cancer, said method comprising administering a therapeutically effective dose of lonidamine or a lonidamine analog in combination with a HIF-1alpha inhibitor.

18. A method of treating cancer, said method comprising administering a therapeutically effective dose of lonidamine or a lonidamine analog in combination with VegF inhibitor.

19. The method of claim 18, wherein said VegF inhibitor is Avastin.

20. The method of claim 19, wherein said cancer is a cancer selected from the group consisting of colon cancer, pancreatic cancer, and renal cell carcinoma.
